(19) United States
(12) Patent Application Publication
(10) Pub. No.: US 2009/0281040 A1
(43) Pub. Date: Nov. 12, 2009

(54) Methods For Treating Endoplasmic Reticulum (ER) Stress Disorders

(76) Inventors: Fumihiko Urano, Wellesley, MA (US); Shinsuke Ishigaki, Shrewsbury, MA (US); Sonya G. Fonesca, Worcester, MA (US)

Correspondence Address: FISH & RICHARDSON PC, P.O. BOX 1022, MINNEAPOLIS, MN, 55440-1022, US

(21) Appl. No.: 12/463,225

(22) Filed: May 8, 2009

Related U.S. Application Data
(60) Provisional application No. 61/051,608, filed on May 8, 2008.

(51) Int. Cl. A61K 38/17; C12Q 1/26; A61K 31/713; A61P 7/00; A61P 3/10

(52) U.S. Cl. 514/12; 435/25; 514/44 A

(57) ABSTRACT

The present invention provides methods for treating ER stress disorders and for identifying compounds for treating ER stress disorders.

### CROSS-REFERENCE TO RELATED APPLICATIONS

This application claims the benefit of the filing date of U.S. Provisional Application No. 61/051,608, which was filed May 8, 2008. This prior application is hereby incorporated by reference herein in its entirety.

### TECHNICAL FIELD

This invention relates to treatment for ER stress disorder.

### BACKGROUND

The endoplasmic reticulum (ER) is a multi-functional cellular compartment that functions in protein folding, lipid biosynthesis, and calcium homeostasis. Perturbations in ER function cause dysregulation of ER homeostasis and accumulation of misfolded and unfolded proteins in the organelle, leading to ER stress. Cells cope with ER stress by activating an ER stress signaling network, also called the Unfolded Protein Response (UPR) (see, e.g., D. Ron, P. Walter, Nat Rev Mol Cell Biol 8, 519 (2007); D. T. Rutkowski, R. J. Kaufman, Trends Biochem Sci (2007)). The UPR consists of three components that counteract ER stress: gene expression, translational attenuation, and ER-associated protein degradation (the ERAD system) (Harding et al., Ann. Rev. Cell Dev. Biol. 18:575-599 (2002); Kaufman et al., Nat. Rev. Mol. Cell. Biol. 3:411-421 (2002); Mori, Cell 101:451-454 (2000)).

Proteins destined for secretion such as insulin and alpha1-antitrypsin are translocated into the ER co-translationally; once there, they undergo highly ordered protein folding and post-translational protein processing. However, in some instances, the sensitive folding environment in the ER can be perturbed by pathophysiological processes such as viral infections, environmental toxins, and mutant protein expression, as well as natural processes such as the large biosynthetic load placed on the ER. When the demand that the load of proteins makes on the ER exceeds the actual folding capacity of the ER to meet that demand, a condition termed “ER stress” results.

Evidence suggest that chronic ER stress is of major importance in the pathogenesis of diabetes mellitus, as well as neuronal disorders such as Parkinson's disease, amyotrophic lateral sclerosis, and mental disorders (see, e.g., Smith, W. W. et al., Hum Mol Genet. 14, 3801-3811 (2005); Cooper, A. A., et al., Science (New York, N.Y. 313, 324-328 (2006); Lipson et al., Curr Mol Med 6, 71-77 (2006); Fonseca, S. G., et al., The Journal of Biological Chemistry 280, 39609-39615 (2005); Atkin, J. D., et al., The Journal of Biological Chemistry 281, 30152-30165 (2006); Turner, B. J. & Atkin, J. D., Curr Mol Med 6, 79-86 (2006); Holtz, W. A. & O'Malley, K. L., The Journal of Biological Chemistry 278, 19367-19377 (2003); Ryu, E. J., et al. J Neurosci 22, 10690-10698 (2002); Uehara, T., et al., Nature 441, 513-517 (2006). In such diseases, the dysregulation of ER homeostasis leads to cellular dysfunction and the activation of cell-death pathways.

### SUMMARY

In one aspect, the invention provides an isolated insulin-producing cell, wherein the cell is an exocrine pancreatic cell comprising an exogenous nucleic acid that encodes a WFS1 polypeptide, and expressing an amount of the WFS1 polypeptide sufficient to induce the cell to secrete insulin.

In another aspect, provided herein are methods for making an insulin-producing cell, the methods comprising providing an exocrine pancreatic cell, and up-regulating the expression of a WFS1 polypeptide in the cell. In some instances, the expression of the WFS1 polypeptide is up-regulated in the cell by introducing into the cell a nucleic acid molecule comprising a nucleic acid sequence encoding WFS1. In other instances, the nucleic acid molecule is a viral vector.

In another aspect, described herein are methods for treating diabetes in a patient, the methods comprising: (a) obtaining an exocrine pancreatic cell; (b) up-regulating the expression of a WFS1 polypeptide in the cell such that the cell produces insulin; and (c) introducing the insulin-producing cell into the patient. In some instances, the exocrine pancreatic cell is derived from the patient to be treated.

In yet another aspect, the invention provides methods for treating diabetes in a patient, the methods comprising: (a) obtaining a nucleic acid molecule comprising a nucleic acid sequence encoding a WFS1 polypeptide; and (b) introducing the nucleic acid molecule into the pancreas of the patient, such that the WFS1 polypeptide is expressed in the exocrine pancreatic cells of the patient, enabling the cells to produce insulin.

In another aspect, provided herein are methods for inhibiting cell death of a cell under ER stress, the methods comprising administering to the cell a nucleic acid molecule comprising a nucleic acid sequence encoding an Apoptosis Antagonizing Transcription Factor (“AATF”), an AATF polypeptide, or functional fragment thereof.

In one aspect, the invention provides methods for treating an ER stress disorder in a patient, the methods comprising administering to the patient a therapeutically effective amount of a nucleic acid molecule comprising a nucleic acid sequence encoding AATF, an AATF polypeptide, or functional fragment thereof.

In another aspect, described herein are methods for identifying a candidate compound for modulating ER stress signaling the methods comprising: (a) obtaining an ER stress model system; (b) contacting the model system with a test compound; and (c) comparing the expression level or activity of AATF in the model system in the presence and in the absence of the test compound; wherein increased AATF expression level or activity in the presence of the test compound indicates that the test compound is a candidate compound for reducing ER stress signaling, and wherein decreased AATF expression level or activity in the presence of the test compound indicates that the test compound is a candidate compound for increasing ER stress signaling.

In one aspect, provided herein are methods for identifying a candidate compound for modulating ER stress signaling, the method comprising: (a) obtaining a cell that expresses an AATF polypeptide and comprises a nucleic acid molecule comprising an Akt1 promoter region operably linked to a reporter gene; (b) contacting the cell with a test compound; and (c) compare the expression level of the reporter gene in the presence and in the absence of the compound; wherein an increase in the expression level in the presence of the compound indicates that the test compound is a candidate compound for reducing ER stress signaling and a decrease in the expression level in the presence of the compound indicates that the test compound is a candidate compound for increasing ER stress signaling.

In yet another aspect, the invention provides methods for identifying a candidate compound for modulating ER stress signaling, the methods comprising: (a) obtaining a first polypeptide that: (i) comprises a WFS1 protein or a fragment thereof; and (ii) displays ATF6-binding ability; (b) obtaining a second polypeptide that: (i) comprises an ATF6 protein or a fragment thereof; and (ii) displays WFS1-binding ability; (c) contacting the first and second polypeptides in the presence of a test compound; and (d) comparing the level of binding between the first and second polypeptides in the presence of the test compound with the level of binding in the absence of the test compound; wherein a different level of binding in the presence of the test compound than in its absence indicates that the test compound is a candidate compound for modulating ER stress signaling.

In one aspect, described herein are methods for identifying a candidate compound for modulating ER stress signaling, the method comprising: (a) obtaining a first polypeptide that: (i) comprises a WFS1 protein or a fragment thereof; and (ii) displays HRD1-binding ability; (b) obtaining a second polypeptide that: (i) comprises an HRD1 protein or a fragment thereof; and (ii) displays WFS1-binding ability; (c) contacting the first and second polypeptides in the presence of a test compound; and (d) comparing the level of binding between the first and second polypeptides in the presence of the test compound with the level of binding in the absence of the test compound; wherein a different level of binding in the presence of the test compound than in its absence indicates that the test compound is a candidate compound for modulating ER stress signaling.

In another aspect, the invention provides methods for identifying a candidate compound for modulating ER stress signaling, the methods comprising: (a) providing a first polypeptide that: (i) comprises a ATF6 protein or a fragment thereof; and (ii) displays HRD1-binding ability; (b) providing a second polypeptide that: (i) comprises an HRD1 protein or a fragment thereof; and (ii) displays ATF6-binding ability; (c) contacting the first and second polypeptides in the presence of a test compound; and (d) comparing the level of binding between the first and second polypeptides in the presence of the test compound with the level of binding in the absence of the test compound; wherein a different level of binding in the presence of the test compound than in its absence indicates that the test compound is a candidate compound for modulating ER stress signaling.

In yet another aspect, described herein are methods for identifying a candidate compound for modulating ER stress signaling, the methods comprising: (a) obtaining an ER stress model system; (b) contacting the model system with a test compound; and (c) comparing the level of binding between WFS1 protein and ATF6 protein in the model system in the presence and in the absence of the test compound; wherein a different level of binding in the presence of the test compound than in its absence indicates that the test compound is a candidate compound for modulating ER stress signaling.

In one aspect, the invention provides methods for identifying a candidate compound for modulating ER stress signaling, the method comprising: (a) obtaining an ER stress model system; (b) contacting the model system with a test compound; and (c) comparing the level of binding between WFS1 protein and HRD1 protein in the model system in the presence and in the absence of the test compound; wherein a different level of binding in the presence of the test compound than in its absence indicates that the test compound is a candidate compound for modulating ER stress signaling.

In another aspect, provided herein are methods for identifying a candidate compound for modulating ER stress signaling, the methods comprising: (a) obtaining an ER stress model system; (b) contacting the model system with a test compound; and (c) comparing the level of binding between HRD1 protein and ATF6 protein in the model system in the presence and in the absence of the test compound; wherein a different level of binding in the presence of the test compound than in its absence indicates that the test compound is a candidate compound for modulating ER stress signaling.

In yet another aspect, described herein are methods for identifying a candidate compound for modulating ER stress signaling, the method comprising: (a) obtaining an ER stress model system; (b) contacting the model system with a test compound; and (c) comparing the level of a protein complex comprising WFS1, ATF6 and HRD1 in the model system in the presence and in the absence of the test compound; wherein a different level of the protein complex in the presence of the test compound than in its absence indicates that the test compound is a candidate compound for modulating ER stress signaling.

In one aspect, provided herein are methods for determining a subject's risk of developing a condition associated with ER stress-related cell death, the method comprising: providing a sample comprising a cell from the subject; determining levels of one or both of HRD1 and ATF6 protein, or cellular localization of ATF6 protein in the sample; and comparing the levels of one or both of HRD1 and ATF6 protein, or cellular localization of ATF6 protein in the sample with the corresponding levels of HRD1 and ATF6 protein, or cellular localization of ATF6 protein, in a control sample; wherein a difference in the level of HRD1 or ATF6 protein, or cellular localization of ATF6, in the test sample as compared to the control sample indicates the subject's risk of developing a condition associated with ER stress-related cell death.

In another aspect, provided herein are methods of treating a subject having a condition associated with ER stress-related cell death, the method comprising: selecting a subject in need of such treatment; and administering to the subject a therapeutically effective amount of one or more of: an HRD1 agonist, e.g., an HRD1 protein, or a nucleic acid sequence encoding HRD1 protein; or an ATF6-specific inhibitory nucleic acid or antagonist, thereby treating the subject.

In another aspect, provided herein are methods for identifying a candidate compound to treat a condition associated with ER stress-related cell death, the method comprising: providing a cell expressing HRD1 and ATF6, wherein the cell expresses no or little WFS1 protein; exposing the cell to a test compound; and comparing protein levels of HRD1 and ATF6 in the cell in the presence of the test compound with levels of HRD1 and ATF6 in the absence of the test compound; wherein a higher level of HRD1 or a lower level of ATF6 in the presence of the test compound than in its absence indicates that the test compound is a candidate compound for treating a disorder associated with ER stress-related cell death.

In yet another aspect, provided herein are methods for identifying a candidate compound for reducing ER stress-induced signaling, the method comprising: providing a sample comprising HRD1 and ATF6 proteins; contacting the sample with a test compound; and comparing binding between HRD1 and ATF6 in the presence of the test compound with binding between HRD1 and ATF6 in the absence of the test compound; wherein a higher level of binding in the presence of the test compound than in its absence indicates that the test compound is a candidate compound for reducing ER stress signaling.

### DESCRIPTION OF DRAWINGS

FIG. 1A is a set of three bar graphs showing that AATF mRNA was up-regulated by tunicamycin (TM), thapsigargin (Tg), and MG132, but not by a general apoptosis inducer, staurosporin. INS1 832/13 cells, Neuro2a (N2a) cells, and mouse embryonic fibroblasts (MEF) were challenged to various ER stress inducers. INS1 832/13 cells were treated with thapsigargin (Tg, 1 μM) and MG132 (2 μM) for 16 hr. Neuro2a (N2a) cells and mouse embryonic fibroblasts (MEF) were treated with tunicamycin (TM, 5 μg/ml) and thapsigargin (Tg, 1 μM) for 16 hr. Cells were also treated with staurosporin (STR, 0.05 μM and 0.01 μM) for 16 hr or untreated. Expression levels of Aatf were measured by real-time PCR. (n=3; values are mean±SD).

FIG. 1B is a reproduction of immunoblots showing that AATF expression was up-regulated by ER stress in both cytoplasmic and nuclear protein extracts from INS-1 832/13 cells. INS-1 832/13 cells were treated with thapsigargin (Tg, 1 μM) for the indicated periods. Expression levels of Aatf and Creb were measured by immunoblot using cytoplasmic and nuclear extracts.

FIG. 1C is a set of five bar graphs showing expression level of AATF (Aatf) as compared to other ER stress markers, including BiP, Chop, XBP-1, and WFS1, in cells treated with thapsigargin. INS1 832/13 cells were treated with thapsigargin (Tg, 0.5 μM) for the indicated times. Expression levels of Aatf, Wfs1, Chop, BiP, and total and spliced Xbp-1 mRNA were measured by real-time PCR (n=3; values are mean±SD).

FIGS. 2A and 2B are bar graphs showing expression levels of AATF in Ire1α^{31 /-} and Perk^{-/-} mouse embryonic fibroblasts under ER stress conditions. (A) Wild-type (Wt), Ire1α^{-/-}, and Perk^{-/-} mouse embryonic fibroblasts were treated with three ER stress inducers, tunicamycin (TM, 5 μg/ml) and thapsigargin (Tg, 1 μM) for 16 hr. Cells were also treated with staurosporin (STR, 0.05 μM and 0.01 μM) for 16 hr or untreated. Expression levels of AATF were measured by real-time PCR. (n=3; values are mean±SD). (B) Wild type (Wt), Ire1α^{-/-}, and Perk^{-/-} mouse embryonic fibroblasts were treated with thapsigargin (Tg, 1 μM) at different times. Expression levels of Aatf were measured by real-time PCR. (n=3; values are mean±SD).

FIG. 2C is a pair of bar graphs (upper panel) showing expression level of AATF in wild-type and Perk^{-/-} mouse fibroblasts treated with salubrina, and a reproduction of an immunoblot (lower panel) that eIF2α phosphorylation levels were increased by salubrina. Wild-type (Wt) and Perk^{-/-} mouse embryonic fibroblasts were treated with thapsigargin (Tg, 1 μM) or Salubrinal (75 nM) for 16 hr. Expression levels of Aatf (left panel) and Chop (right panel) were measured by real-time PCR. (n=3; values are mean±SD) Expression levels of phosphorylated eIf2α and actin were measured by immunoblot (lower panel).

FIG. 2D is a set of three bar graphs showing that reconstitution of Perk in Perk^{-/-} mouse embryonic fibroblasts recovered AATF gene expression. Perk^{-/-} mouse embryonic fibroblasts were transfected with pcDNA3/Perk and then treated with or without thapsigargin (Tg, 1 μM) for 8 hr. Expression levels of Aatf, Chp, and Perk mRNA were measured by real-time PCR (n=3; values are mean±SD).

FIG. 3A is a pair of immunoblots showing the results from transfecting siRNA directed against AATF in INS-1 832/13 cells, then challenging the cells with thapsigargin or staurosporin, and measuring the cleavage of caspase-3, a marker for apoptosis. INS1 832/13 cells were transfected with control scramble siRNA or siRNA against AATF, then treated with two different concentrations of thapsigargin (Tg) (left panel) or staurosporin (STR) (right panel) for 24 hr. Expression levels of caspase-3 (Casp3), AATF, and actin were measured by immunoblot. Single and double asterisks indicate uncleaved and cleaved caspase-3, respectively.

FIG. 3B is a bar graph showing results of measuring apoptosis in AATF-knockdown cells using TUNEL staining. INS1 832/13 cell were transfected with control scramble siRNA or siRNA against AATF, then treated with three different concentrations (0, 0.25, and 0.5 μM) of thapsigargin (Tg) for 24 hr. Apoptotic cells were detected by TUNEL staining. Three independent experiments were carried out and TUNEL-positive cells were counted blindly (n=3; values are mean±SD). Statistics were done by two-way ANOVA. *(p<0.01) denotes significant differences between cells transfected with control scramble siRNA and siRNA against AATF.

FIG. 3C is a reproduction of an immunoblot (upper panel) showing that AATF induction decreased caspase-3 cleavage in cells treated with thapsigargin, and a bar graph (lower panel) showing that AATF induction decreased the number of TUNEL-positive cells. INS-1 832/13 cells were stably transduced with pLenti-TO/AATF, inducible lentivirus expressing AATF. Cells were cultured with doxycycline (2 μg/ml) to induce AATF or without doxycycline for 48 hr, then challenged with thapsigargin (Tg, 0.5 μM) for 16 h. Expression levels of caspase-3 (Casp3), AATF, and actin were measured by immunoblot. Single and double asterisks indicate uncleaved and cleaved caspase-3, respectively. The ratio between cleaved caspase-3 and actin was measured using ImageJ software (upper panel). Cells were cultured with doxycycline (2 μg/ml) to induce AATF (AATF O/E) or without doxycycline (Cont) for 48 hr, then challenged with three different concentrations of thapsigargin (0, 0.5, and 1.0 μM) of thapsigargin (Tg) for 24 hr. Apoptotic cell death was assessed by the TUNEL assay. Three independent experiments were carried out (n=3; values are mean±SD) Statistics were done by two-way ANOVA. *(p<0.01) denotes significant differences between cells with and without doxycycline. (lower panel).

FIG. 3D is a bar graph and a reproduction of immunoblot showing results from culturing INS-1 832/13 cells in glucose-free medium, then measuring expression levels of Chop and AATF, as well as capase-3 cleavage. Glucose deprivation and α-synuclein expression induce ER stress-mediated apoptosis. Glucose deprivation causes ER stress-mediated apoptosis. INS-1 832/13 cells were cultured in glucose-free media for the indicated times. Expression levels of Chop and AATF mRNA were measured by real-time PCR (left panel) (n=3; values are mean±SD). Expression levels of caspase-3 (Casp3) and actin were measured by immunoblot. Single and double asterisks indicate uncleaved and cleaved caspase-3, respectively (right panel).

FIG. 3E is a reproduction of immunoblot showing that AATF-knockdown sensitized INS-1 832/13 cells to glucose deprivation-mediated apoptosis. INS-1 832/13 cells were transfected with control scramble siRNA (Control) or siRNA against AATF, then cultured in glucose-free media for 48 hr. Expression levels of caspase-3 (Casp3), AATF, and actin were measured by immunoblot. Single and double asterisks indicate uncleaved and cleaved caspase-3, respectively.

FIG. 3F is a reproduction of immunoblot showing that AATF over-expression using doxycycline-mediated induction decreased caspase-3 cleavage caused by glucose deprivation in INS-1 832/13 cells. INS1 . 832/13 cells were stably transduced with pLenti-TO/AATF, inducible lentivirus expressing AATF. Cells were cultured with doxycycline (2 μg/ml) to induce AATF or without doxycycline (2 μg/ml) for 48 hr, then cultured in glucose-free media for 48 hr. Expression levels of caspase-3 (Casp3), AATF, and actin were measured by immunoblot. Single and double asterisks indicate uncleaved and cleaved caspase-3, respectively.

FIG. 3G is bar graphs showing the results of measuring expression levels of AATF, CHOP and BIP mRNA in SH-SY5Y cells over-expressing α-synuclein. Expression levels of Aatf, Chop, and BiP mRNA were measured by real-time PCR (n=3; values are mean±SD).

FIG. 3H is a reproduction of an immunoblot showing that that eIF2α phosphorylation was increased in SH-SY5Y cells expressing α-synuclein.

FIG. 3I are bar graphs showing results from transfecting SH-SY5Y cells expressing α-synuclein with siRNA directed against AATF, then measuring cell viability and death. Suppression of AATF expression decreased viability (left panel) and increased apoptosis (right panel) in the cells expressing α-synuclein as compared to control cells. SH-SY5Y cells stably and constitutively expressing α-synuclein (αSyn) or GFP were transfected with control scramble siRNA (Control) or siRNA against AATF. After overnight incubation, MTS assays (Left panel) and cell toxicity assays (Right panel) were performed. Values are the means±SD, n=6. Statistics were done by two-way ANOVA. *(p<0.01) denotes significant differences between cells transfected with control scramble siRNA and siRNA against AATF.

FIG. 3J is a reproduction of an immunoblot showing that AATF-knockdown increased the cleavage of caspase-3 in SH-SY5Y cells expressing α-synuclein, but not in control cells. SH-SY5Y cells stably and constitutively expressing α-synuclein (αSyn) or GFP were transfected with control scramble siRNA (Control) or siRNA against AATF, then cultured for 24 hr. Expression levels of caspase-3 (Casp3), AATF, α-synuclein (ccSyn), and tubulin and were measured by immunoblot. Single and double asterisks indicate uncleaved and cleaved caspase-3, respectively.

FIG. 4A is a bar graph and a reproduction of an immunoblot showing that AATF-knockdown by siRNA suppressed Akt1 mRNA and protein expression. INS1 832/13 cells were transfected with scramble siRNA (control) or siRNA against AATF. Expression levels of AATF mRNA were measured by real-time PCR (n=3; values are mean±SD) (upper panel). Expression levels of total AKT (AKT), AKT1, AATF, and actin were measured by immunoblot using cell extracts from INS1 . 832/13 cells (lower panel).

FIG. 4B is a set of bar graphs showing that Akt1 mRNA expression was increased 1.5-2 fold by various ER stress inducers, including tunicamycin, thapsigargin, and MG132, but not staurosporin. INS1 . 832/13 cells, Neuro2a (N2a) cells, and mouse embryonic fibroblasts (MEF) were challenged to various ER stress inducers. INS1 832/13 cells were treated with thapsigargin (Tg, 1 μM) and MG132 (2 μM) for 16 hr. Neuro2a (N2a) cells and mouse embryonic fibroblasts (MEF) were treated with tunicamycin (TM, 5 μg/ml) and thapsigargin (Tg, 1 μM) for 16 hr. Cells were also treated with staurosporin (STR, 0.05 μM and 0.01 μM) for 16 hr or untreated. Expression levels of Akt1 were measured by real-time PCR. (n=3; values are mean±SD).

FIG. 4C is a bar graph (left panel) showing that Akt1 mRNA expression was increased during ER stress with a peak at 24 hr, and a reproduction of an immunoblot (right panel) showing that the phosphorylation level of Akt was increased up to 8 hr after thapsigargin treatment, but decreased at 24 hr. INS1 832/13 cells were treated with thapsigargin (Tg, 1 μM) for the indicated times. Expression levels of Akt1 mRNA were measured by real-time PCR (n=3; values are mean±SD) (left panel). Expression levels of phosphorylated AKT (P-AKT), total AKT (AKT), and actin were also measured by immunoblot (right panel).

FIG. 4D is a reproduction of an immunoblot showing the results from using siRNA directed against AATF in INS-1 832/13 cells and treating the cells with thapsigargin for 0, 3, and 8 hr, then measuring Akt expression and Akt phosphorylation levels. INS1 832/13 cells were transfected with scramble siRNA (control) or siRNA against AATF, then treated with thapsigargin (Tg) (0.5 MlM) for the indicated times. Expression levels of phosphorylated AKT (P-AKT), total AKT (AKT), AATF, and actin were measured by immunoblot.

FIG. 4E is a bar graph and a reproduction of an immunoblot showing that AATF over-expression enhanced Akt1 mRNA expression under ER stress conditions, leading to an increase in Akt phosphorylation. INS1 832/13 cells were stably transduced with pLenti-TO/AATF, inducible lentivirus expressing AATF. Cells were cultured with or without doxycycline (Dox, 2 μg/ml) to induce AATF for 48 hr, then challenged with thapsigargin (Tg, 0.5 μM) for 16 hr. Expression levels of Akt1 mRNA were measured by real-time PCR (n=3; values are mean±SD) (left panel). Expression levels of phosphorylated AKT (P-AKT), AATF, and actin were also measured by immunoblot (right panel).

FIGS. 4F and 4G are bar graphs showing the results of co-transfecting a plasmid expressing Stat3 with or without AATF into 293T cells along with a reporter plasmid containing 1.3 kilobases of the Akt1 promoter driving the luciferase gene. (F) The promoter activity of Akt1 was measured using pGL4.14/Akt1^{-1323/-1} co-expressed with the combination of pFlag/STAT3-C(STAT3), pCS2+/AATF (AATF), and siRNA against AATF. N2a cells were transfected with β-galactosidase and constructs indicated in the figure. Following a 24 hour culture, luciferase activity was measured and normalized to β-galactosidase. The ratio of pGL4.14/Akt^{-1323/-1} to pGL4.14/mock was indicated (n=3; values are mean±SD). (G) Quantified ChIP analysis using real-time PCR was performed. Relative recruitment was defined as the ratio of amplification of the PCR product relative to 1% of input genomic DNA. Value obtained from mock was defined as 1. (n=3; values are mean±SD).

FIG. 4H is a reproduction of an immunoblot showing that Stat3 and Akt1 interact in the nucleus. Nuclear fraction of HEK293 cells were extracted and applied for immunoprecipitation using anti-AATF antibody. Immunoprecipitated samples and 5% inputs were blotted with indicated antibodies.

FIG. 4I is a reproduction of immunoblots showing the result of using siRNA directed against Akt1 (left panel) or an Akt inhibitor, SH-5 (right panel), against INS1 832/13 cells, and challenging these cells with thapsigargin and measuring the cleavage of caspase-3. INS1 832/13 cells were transfected with control scramble siRNA or siRNA against Akt1, then treated with 0.25 μM of thapsigargin (Tg) for 16 hr (left panel). INS1 832/13 cells were pretreated with 10 nM of Akt inhibitor (SH-5) or equivalent amount of DMSO (control) for overnight, then treated with 0.25 μM of thapsigargin (Tg) for 16 hr (right panel). Expression levels of caspase-3 (Casp3), phosphorylated AKT (P-AKT), total AKT (AKT), and actin were measured by immunoblot. Single and double asterisks indicate uncleaved and cleaved caspase-3, respectively.

FIG. 4J is a reproduction of immunoblots showing the results from blocking the Akt1 pathway in INS1 832/13 cells using an Akt inhibitor, SH-5, then challenging the cells with glucose deprivation, and measuring the cleavage of caspase-3. INS-1 832/13 cells were pretreated with 10 nM of Akt inhibitor (SH-5) or equivalent amount of DMSO for overnight, then cultured in glucose-free media for 48 hr. Expression levels of caspase-3 (Casp3), phosphorylated AKT (P-AKT), total AKT (AKT), and actin were measured by immunoblot. Single and double asterisks indicate uncleaved and cleaved caspase-3, respectively.

FIG. 4K is a reproduction of immunoblots showing the results from transfecting INS-1 832/13 cells with control siRNA or siRNA against AATF, then challenging these cells with or without the induction of Akt1, using the lentivirus-based doxycycline-mediated Akt1 induction system, and measuring caspase-3 cleavage. INS-1 832/13 cells were stably transduced with pLenti-TO/Akt1, inducible lentivirus expressing active form of Akt1. Cells were cultured with doxycycline (4 ng/ml) to induce Akt1 or without doxycycline (4 ng/ml) for 48 hr, then challenged with thapsigargin (Tg, 0.5 μM) for 16 hours. Cells were also transfected with control scramble siRNA (Cont) or siRNA against AATF. Expression levels of caspase-3 (Casp3), total AKT (AKT), phosphorylated AKT (P-AKT), AATF, and actin were measured by immunoblot. Single and double asterisks indicate uncleaved and cleaved caspase-3, respectively.

FIG. 5A is a reproduction of immunoblots showing expression of WFS1 in INS-1 832/13, transduced with an inducible lentivirus expressing human WFS1.

FIG. 5B are bar graphs showing expression levels of BiP, total Xbp-1, Chop, Ero-1α, Glut2, and Ins2 in INS-1 832/13 cells over-expressing GFP (control) or WFS1.

FIG. 5C is a set of bar graphs showing the results from transfecting COS7 cells with ATF6 expression plasmid or ATF6 and WFS1 expression plasmids together with the following luciferase reporter genes: ATF6 binding site reporter gene ATF6GL3 (left panel), ATF6 mutant site reporter gene ATF6m1GL3 (middle panel), or rat GRP78 promoter reporter gene ERSE (right panel). Relative intensity of luciferase was then measured (n=3).

FIG. 6A is a reproduction of immunoblots showing that WFS1 associated with ATF6 under non-stress conditions (left panel), and that DTT treatment of INS-1 832/13 cells caused a dissociation of ATF6 from WFS1 in a time-dependent manner, with almost complete dissociation 3 hours post-treatment (right panel).

FIG. 6B is a reproduction of immunoblots showing that the interaction of ATF6 and WFS1 in INS1 832/13 cells began to recover after a 3 hour chase in normal media following 2 hours of treatment with DTT.

FIG. 7A is a reproduction of immunoblots showing that ATF6 protein level in INS1 832/13 cells expressing WFS1 was reduced by more than 2-fold.

FIG. 7B is a reproduction of immunoblots showing that ATF6 protein levels in MIN6 expressing shRNA against WFS1 were increased approximately 2-fold compared to control MIN6 cells expressing shRNA directed against GFP (left panel), and that ATF6 protein expression levels were again reduced when WFS1 was reintroduced (right panel).

FIG. 7C is a reproduction of immunoblots showing that when WFS1 is expressed with ATF6 in a 1:1 ratio in COS-7 cells, the steady-state level of ATF6 protein was reduced by 2-fold, while a 1:2 ratio of ATF6 to WFS1 almost abolished ATF6 protein levels (left panel), and that treatment with MG132 led to an almost full recovery of ATF6 protein levels (right panel).

FIG. 7D is a reproduction of an immunoblot and a graph showing that co-transfection of WFS1 with ATF6 in COS-7 cells decreased ATF6 protein expression levels as compared to control.

FIG. 7E is a reproduction of immunoblots showing that when endogenous ATF6 was immunoprecipitated from INS-1 832/13 cells infected with lentivirus expressing human WFS1 or GFP and then treated with the proteosome-inhibitor, MG132, there was a marked enhancement of ATF6 ubiquitination in cells expressing WFS1.

FIG. 8A is a reproduction of immunoblots showing the results of immunoprecipitating WFS1 from INS 1832/13 cells, and then immunoblotted the IP product with an α-5 proteasome subunit-specific antibody.

FIG. 8B-1, 8B-2, and 8C are reproductions of immunoblots showing results from fractionating purified ER extracts from INS-1 832/13 cells using glycerol gradient sedimentation (FIG. 8B-1). The expression of the 26 S proteasome, ATF6, and WFS1 was found to overlap in fractions 8-13 (FIG. 8B-2). When WFS1 was immunoprecipitated from fractions 10-11, an interaction was found between WFS1 and ATF6, as well WFS1 and the proteasome (FIG. 8C, left panel). When ATF6 was immunoprecipitated from a mixture of factions 9 and 12, an ATF6-proteosome complex could be seen (FIG. 5C, right panel).

FIG. 8D is a reproduction of immunoblots showing results from immunoprecipating HRD1 from INS1 . 832/13 lysates, and then immunoblotting the IP product with a WFS1-specific antibody.

FIG. 8E is a reproduction of an immunoblot and a graph showing that co-transfection of HRD1 with ATF6 in 293T cells enhanced ATF6 protein degradation as compared to control cells.

FIGS. 8F and 8G are reproductions of immunoblots showing the results of fractionating purified ER extracts from INS-1 832/13 cells using glycerol gradient sedimentation. ATF6, HRD1, and WFS1 protein expression overlapped in fraction 13 (FIG. 8F). When HRD1 was immunoprecipitated from this fraction, an interaction between ATF6 and HRD1 could be seen (FIG. 8G).

FIG. 9 is a bar graph showing that expressing WFS1 in exocrine pancreatic cells induce these cells to produce insulin.

FIG. 10 is a reproduction of an immunoblot showing the amount of ATF6 and WFS1 in lymphoblast lysates from Wolfram syndrome patients.

FIG. 11A is a reproduction of an immunoblot showing the amount of HRD1 in lymphoblast lysates from Wolfram syndrome patients.

FIG. 11B is a reproduction of an immunoblot showing the amount of WFS1, HRD1, and c-Myc in MIN6 cells (left panel) and INS1 . 832/13 cells (right panel) mock transfected or transfected with Hrd1-Myc expression plasmid.

### DETAILED DESCRIPTION

This invention is based on the discovery of novel components and regulatory mechanisms of the ER stress signaling pathway. Evidence provided herein shows that Apoptosis-antagonizing transcription factor (AATF) protects cells from ER stress-mediated apoptosis through transcriptional regulation of Akt1, a survival kinase. Further, as described herein, evidence demonstrates that Wolfram syndrome 1 (WFS1) and Activating Transcription Factor 6 (ATF6) form a complex with the proteasome and an E3 ligase, hydroxymethylglutaryl reductase degradation 1 (HRD1), on the ER membrane, leading to degradation of ATF6 under non-stress conditions. Evidence provided herein also show that expressing WFS1 in exocrine pancreatic cells, which do not normally express WFS1 or produce insulin, turn them into insulin-producing cells.

Based on these discoveries, the present application provides, inter alia, methods for treating ER stress disorders, e.g., diabetes (including both type 1 and type 2 diabetes) and neurodegenerative disorders, and methods for identifying compounds for treating ER stress disorders.

I. ER Stress Disorders and ER Stress Signaling

As used herein, the term “ER stress disorder” refers to a disease or disorder associated with (e.g., caused by, resulting from, attributed to, or correlated with, at least in part) increased ER stress levels. Exemplary ER stress disorders include diabetes (e.g., type 1 and type 2 diabetes) and some protein conformational diseases. The term “protein conformational disease” (“PCD”) refers to a disease or disorder (e.g., a human disease or disorder) associated with protein misfolding (e.g., caused by, resulting from, attributed to, or correlated with, at least in part, protein misfolding). Exemplary protein conformational diseases include, but are not limited to, those diseases listed in Table 1. Other diseases include inflammatory bowel disease (Crohn disease and ulcerative colitis); and cancers originated from secretory cells (e.g., breast cancer and prostate cancer).

As used herein, the term “condition associated with ER stress-related cell death” refers to a disorder that can be identified by a decrease in HRD1 levels, an increase in ATF6 levels, or an increase in nuclear localization of ATF6 compared to a control sample. The control sample represents a level in a subject with a normal risk of developing a condition associated with ER stress-related cell death.

As used herein, the terms “ER stress signaling” and “Unfolded Protein Response” (“UPR”) refer to cellular responses that are associated with (e.g., caused by, correlated with, or induced by) ER stress. These cellular responses include, but are not limited to, gene expression, protein expression, and protein degradation. Various methodologies described herein include steps that involve determining or comparing levels of ER stress signaling. Methods for determining levels of ER stress are known in the art. For example, methods for measuring ER stress signaling are described in U.S. Pat. Publication No. 20070202544, the contents of which are incorporated herein by reference. Examples 1 and 2 herein also describe exemplary methods for measuring level of ER stress signaling. For example, expression levels of ER stress response genes, e.g., BiP, Chop, and Xbp-1 can be measured.

TABLE 1
Exemplary ER Stress Disorders/Protein Conformational Diseases

| Disease | Protein involved |
|---|---|
| Alzheimer's disease | amyloid-β |
| immunoglobulin light chain amyloidosis | immunoglobulin light chain |
| Parkinson's disease | alpha-synuclein |
| diabetes mellitus type 2 | amylin |
| amyotrophic lateral sclerosis (ALS) | Superoxide dismutase (SOD) |
| haemodialysis-related amyloidosis | L2-microglobulin |
| reactive amyloidosis | amyloid-A |
| cystic fibrosis | cystic fibrosis transmembrane |
| | regulator (CFTR) |
| sickle cell anemia | hemoglobin |
| Huntington's disease | huntingtin |
| Kreutzfeldt-Jakob disease and related | prions (PrP) |
| disorders (prion encephalopathies) | |
| familial hypercholesterolaemia | low density lipoprotein (LDL) |
| | receptor |
| Alpha1-antitrypsin deficiency, | Alpha1-antitrypsin (alpha1-AT) |
| cirrhosis, emphysema | |
| systemic and cerebral hereditary | (ten other proteins) |
| amyloidoses | |
| Wolcott-Rallison syndrome | translation initiation factor |
| | 2-alpha kinase-3 |
| Wolfram syndrome | Wolfram syndrome 1 (WFS1) |

II. Methods for Treating ER Stress Disorders

Described herein are a number of novel therapeutic targets for the treatment of ER stress disorders.

AATF

Evidence described herein demonstrates that AATF can protect cells, e.g., β-cells and neural cells, from ER-stress induced apoptosis. Thus, the invention provides therapeutic methods for treating ER stress disorders in a patient by, e.g., increasing AATF activity or AATF level, and methods for identifying compounds for treating ER stress disorders by screening for compounds that increase AATF activity or levels.

AATF contains an L-zip domain in the N-terminal, followed by two nuclear localization signals in the C-terminal and has been proposed to play a role in transcription.

AATF polypeptides or fragments thereof, and nucleic acids encoding full-length AATF polypeptides or fragments thereof are useful for the therapeutic and screening methods described herein. AATF polypeptides and nucleic acids encoding them are readily obtained by one of ordinary skill in the art without undue experimentation. For example, the amino acid and nucleic acid sequences of human AATF are known (see, e.g., GenBank Accession No. AF083208.1 for a nucleic acid sequence and GenBank Accession No. AAD52016.1 for an amino acid sequence). A nucleic acid encoding a mammalian, e.g., human, AATF amino acid sequence can be amplified from human cDNA by conventional PCR techniques, using primers upstream and downstream of the coding sequence. AATF cDNAs are also available commercially from, for example, Open Biosystems (Huntsville, Ala.).

HRD1/ATF6

Also described herein is evidence that an increase in HRD1 levels, a decrease in ATF6 levels, or a decrease in nuclear localization of ATF6, can protect cells, e.g., β-cells and neural cells, from ER-stress induced apoptosis. Thus, the invention provides therapeutic methods for treating ER stress disorders in a patient by, e.g., increasing HRD1 activity or HRD1 level, decreasing ATF6 activity or ATF6 level, or decreasing nuclear localization of ATF6, and methods for identifying compounds for treating ER stress disorders by screening for compounds that increase HRD1 activity or levels, decrease ATF6 activity or levels, or decrease nuclear localization of ATF6.

WFS1, ATF6, and HRD1 polypeptides or biologically active fragments thereof, and nucleic acids encoding full-length WYTS1, ATF6, or HRD1 polypeptides or biologically active fragments thereof are useful for the methods described herein. WFS1, ATF6, and HRD1 polypeptides and nucleic acids encoding them are readily obtained by one of ordinary skill in the art without undue experimentation. For example, the amino acid and nucleic acid sequences of human WFS1 are known (see, e.g., GenBank Acc. No. AF084481.1 for a nucleic acid sequence and GenBank Acc. No. 076024.1 for an amino acid sequence). Human HRD1 amino acid and nucleic acid sequences are also known (e.g., Genbank Acc. No. NP_{—}115807.1 or NP_{—}757385.1). Further, human ATF6 amino acid and nucleic acid sequences are known (see, e.g., Genbank Ace. No. AB015856.1 or P18850.3). A nucleic acid encoding a mammalian, e.g., human, WFS1, ATF6 or HRD1 amino acid sequences can be amplified from human cDNA by conventional PCR techniques, using primers upstream and downstream of the coding sequence. WFS1, ATF6 and HRD1 polypeptides or fragments thereof can be produced and isolated using methods described herein.

The terms “patient” is used throughout the specification to describe an animal, human or non-human, rodent or non-rodent, to whom treatment according to the methods of the present invention is provided. Veterinary and non-veterinary applications are contemplated. The term includes, but is not limited to, birds, reptiles, amphibians, and mammals, e.g., humans, other primates, pigs, rodents such as mice and rats, rabbits, guinea pigs, hamsters, cows, horses, cats, dogs, sheep and goats. Typical patients include humans, farm animals, and domestic pets such as cats and dogs.

The term “isolated nucleic acid” means a DNA or RNA that is not immediately contiguous with both of the coding sequences with which it is immediately contiguous (one on the 5′ end and one on the 3′ end) in the naturally occurring genome of the organism from which it is derived. The term includes, for example, recombinant DNA that is incorporated into a vector, into an autonomously replicating plasmid or virus, or into the genomic DNA of a prokaryote or eukaryote, or which exists as a separate molecule (e.g., a genomic DNA fragment produced by PCR or restriction endonuclease treatment) independent of other sequences. It also includes a recombinant DNA that is part of a hybrid gene encoding an additional polypeptide sequence.

The term “purified” refers to a nucleic acid or polypeptide that is substantially free of cellular or viral material with which it is naturally associated, or culture medium (when produced by recombinant DNA techniques), or chemical precursors or other chemicals (when chemically synthesized). Moreover, an isolated nucleic acid fragment is a nucleic acid fragment that is not naturally occurring as a fragment and would not be found in the natural state.

One method for producing polypeptides for use in a method as described herein is recombinant production, which involves genetic transformation of a host cell with a recombinant nucleic acid vector encoding a polypeptide of interest, e.g., AATF or HRD1, expression of the recombinant nucleic acid in the transformed host cell, and collection and purification of the polypeptide. Guidance concerning recombinant DNA technology can be found in numerous well-known references, including Sambrook et al., 1989, Molecular Cloning—A Laboratory Manual, Cold Spring Harbor Press; and Ausubel et al. (eds.), 1994, Current Protocols in Molecular Biology, John Wiley & Sons, Inc.

Purification of recombinant polypeptides can be performed by conventional methods and is within ordinary skill in the art. The purification can include two or more steps, and one step can be affinity chromatography employing antibodies covalently linked to a solid phase chromatography support (beads) such as crosslinked agarose or polyacrylamide. Antibodies are available commercially, for example, from Abcam, Inc. (Cambridge, Mass.) and Sigma-Aldrich (St. Louise, Mo.). Other useful purification steps include gel filtration chromatography and ion exchange chromatography.

Also useful in the methods described herein are genetic constructs (e.g., vectors and plasmids) that include a nucleic acid encoding AATF, HRD1, or ATF6, operably linked to a transcription and/or translation sequence to enable expression, e.g., expression vectors. A selected nucleic acid, e.g., a DNA molecule encoding a polypeptide of interest, is “operably linked” to another nucleic acid molecule, e.g., a promoter, when it is positioned either adjacent to the other molecule or in the same or other location such that the other molecule can direct transcription and/or translation of the selected nucleic acid.

Increasing AATF or HRD1 Activity or Level

Various methods that employ conventional techniques known in the art can be used to increase AATF or HRD1 activity or AATF or HRD1 level in a patient to treat ER stress disorders. For example, an AATF— or HRD1-encoding nucleic acid, polypeptide, or a functional fragment thereof, can be administered to a person having an ER stress disorder such as diabetes, to thereby treat the ER stress disorder. In some instances, compounds that activate AATF or HRD1, e.g., compounds identified from the screening methods described herein, can be administered to increase AATF or HRD1 level or activity.

The AATF or HRD1 polypeptides or AATF— or HRD1-encoding nucleic acids can be administered as part of a pharmaceutical composition, as described herein.

Expression constructs, e.g., a construct that includes a nucleic acid molecule encoding an AATF or HRD1 polypeptide, can be administered in any biologically effective carrier, e.g. any formulation or composition capable of effectively delivering the component gene to cells in vivo. Approaches include insertion of the subject gene in viral vectors including recombinant retroviruses, adenovirus, adeno-associated virus, and herpes simplex virus-1, or recombinant bacterial or eukaryotic plasmids. Viral vectors transfect cells directly; plasmid DNA can be delivered with the help of, for example, cationic liposomes (lipofectin) or derivatized (e.g. antibody conjugated), polylysine conjugates, gramacidin S, artificial viral envelopes or other such intracellular carriers, as well as direct injection of the gene construct or CaPO₄ precipitation carried out in vivo.

An approach for in vivo introduction of nucleic acid into a cell is by use of a viral vector containing nucleic acid, e.g., a cDNA encoding an AATF or HRD1 polypeptide. For example, the inducible lentiviral expression vectors described in Example 1 herein can be used to introduce a nucleic acid encoding AATF into cells. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells which have taken up viral vector nucleic acid.

Retrovirus vectors and adeno-associated virus vectors can be used as a recombinant gene delivery system for the transfer of exogenous genes in vivo, particularly into humans. These vectors provide efficient delivery of genes into cells, and the transferred nucleic acids are stably integrated into the chromosomal DNA of the host. The development of specialized cell lines (termed “packaging cells”) which produce only replication-defective retroviruses has increased the utility of retroviruses for gene therapy, and defective retroviruses are characterized for use in gene transfer for gene therapy purposes (for a review see Miller, A. D. (1990) Blood 76:271). A replication defective retrovirus can be packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are known to those skilled in the art. Examples of suitable packaging virus lines for preparing both ecotropic and amphotropic retroviral systems include *Crip, *Cre, *2, and *Am.

Another viral gene delivery system useful in the present invention utilizes adenovirus-derived vectors. The genome of an adenovirus can be manipulated such that it encodes and expresses a gene product of interest, but is inactivated in terms of its ability to replicate in a normal lytic viral life cycle. See, for example, Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 dl324 or other strains of adenovirus (e.g., Ad2, Ad3, Ad7) are known to those skilled in the art. Recombinant adenoviruses can be advantageous in certain circumstances in that they are not capable of infecting nondividing cells and can be used to infect a wide variety of cell types, including epithelial cells (Rosenfeld et al. (1992) cited supra). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situ where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al. cited supra; Haj-Ahmand and Graham (1986) J. Virol. 57:267).

Yet another viral vector system useful for delivery of the subject gene is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al. (1992) Curr. Topics in Micro. and Immunol. 158:97-129). It is also one of the few viruses that can integrate its DNA into non-dividing cells, and exhibits a high frequency of stable integration (see for example Flotte et al. (1992) Am. J. Respir. Cell. Mol. Biol. 7:349-356; Samulski et al. (1989) J. Virol. 63:3822-3828; and McLaughlin et al. (1989) J. Virol. 62:1963-1973). Vectors containing as little as 300 base pairs of AAV can be packaged and can integrate. Space for exogenous DNA is limited to about 4.5 kb. An AAV vector such as that described in Tratschin et al. (1985) Mol. Cell. Biol. 5:3251-3260 can be used to introduce DNA into cells. A variety of nucleic acids have been introduced into different cell types using AAV vectors (see for example Hermonat et al. (1984) Proc. Natl. Acad. Sci. USA 81:6466-6470; Tratschin et al. (1985) Mol. Cell. Biol. 4:2072-2081; Wondisford et al. (1988) Mol. Endocrinol. 2:32-39; Tratschin et al. (1984) J. Virol. 51:611-619; and Flotte et al. (1993) J. Biol. Chem. 268:3781-3790).

In addition to viral transfer methods, such as those illustrated above, non-viral methods can also be employed to cause expression of an AATF or HRD1 polypeptide in the tissue of an animal. Most nonviral methods of gene transfer rely on normal mechanisms used by mammalian cells for the uptake and intracellular transport of macromolecules. In certain embodiments, non-viral gene delivery systems of the present invention rely on endocytic pathways for the uptake of the subject gene by the targeted cell. Exemplary gene delivery systems of this type include liposomal derived systems, poly-lysine conjugates, and artificial viral envelopes. Other embodiments include plasmid injection systems such as are described in Meuli et al. (2001) J Invest Dermatol. 116(1):131-135; Cohen et al. (2000) Gene Ther 7(22):1896-905; or Tam et al. (2000) Gene Ther 7(21):1867-74.

For example, a gene encoding an AATF or HRD1 polypeptide described herein can be entrapped in liposomes bearing positive charges on their surface (e.g., lipofectins) and (optionally) which are tagged with antibodies against cell surface antigens of the target tissue (Mizuno et al. (1992) No Shinkei Geka 20:547-551; PCT publication WO91/06309; Japanese patent application 1047381; and European patent publication EP-A-43075).

In clinical settings, the gene delivery systems for the therapeutic gene can be introduced into a patient by any of a number of methods, including those familiar in the art. For instance, a pharmaceutical preparation of the gene delivery system can be introduced systemically, e.g. by intravenous injection, and specific transduction of the protein in the target cells occurs predominantly from specificity of transfection provided by the gene delivery vehicle, cell-type or tissue-type expression due to the transcriptional regulatory sequences controlling expression of the receptor gene, or a combination thereof. In other embodiments, initial delivery of the recombinant gene is more limited with introduction into the animal being quite localized. For example, the gene delivery vehicle can be introduced by catheter (see U.S. Pat. No. 5,328,470) or by stereotactic injection (e.g. Chen et al. (1994) PNAS 91: 3054-3057).

The pharmaceutical preparation of the gene therapy construct can consist essentially of the gene delivery system in an acceptable diluent, or can comprise a slow release matrix in which the gene delivery vehicle is imbedded. Alternatively, where the complete gene delivery system can be produced in tact from recombinant cells, e.g. retroviral vectors, the pharmaceutical preparation can comprise one or more cells which produce the gene delivery system.

Inhibitory Nucleic Acids, e.g., siRNA, Antisense, Ribozymes, or Aptamers, Directed Against ATF6

The methods described herein can include the use of inhibitory nucleic acids that specifically target ATF6.

RNA Interference

RNA interference (RNAi) is a process whereby double-stranded RNA (dsRNA) induces the sequence-specific regulation of gene expression in animal and plant cells and in bacteria (Aravin and Tuschl, FEBS Lett. 26:5830-5840 (2005); Herbert et al., Curr. Opin. Biotech. 19:500-505 (2008); Hutvagner and Zamore, Curr. Opin. Genet. Dev.: 12, 225-232 (2002); Sharp, Genes Dev., 15:485-490 (2001); Valencia-Sanchez et al. Genes Dev. 20:515-524 (2006)). In mammalian cells, RNAi can be triggered by 21-nucleotide (nt) duplexes of small interfering RNA (siRNA) (Chiu et al., Mol. Cell. 10:549-561 (2002); Elbashir et al., Nature 411:494-498 (2001)), by microRNA (miRNA), functional small-hairpin RNA (shRNA), or other dsRNAs which are expressed in vivo using DNA templates with RNApolymerase II or III promoters (Zeng et al., Mol. Cell. 9:1327-1333 (2002); Paddison et al., Genes Dev. 16:948-958 (2002); Denti, et al., Mol. Ther. 10:191-199 (2004); Lee et al., Nature Biotechnol. 20:500-505 (2002); Paul et al., Nature Biotechnol. 20:505-508 (2002); Rossi, Human Gene Ther. 19:313-317 (2008); Tuschl, T., Nature Biotechnol. 20:440-448 (2002); Yu et al., Proc. Natl. Acad. Sci. USA 99(9):6047-6052 (2002); McManus et al., RNA 8:842-850 (2002); Scherer et al., Nucleic Acids Res. 35:2620-2628 (2007); Sui et al., Proc. Natl. Acad. Sci. USA 99(6):5515-5520 (2002)).

siRNA Molecules

In general, the methods described herein can use dsRNA molecules comprising 16-30, e.g., 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a target region in the mRNA, and the other strand is complementary to the first strand. The dsRNA molecules can be chemically synthesized, or can transcribed be in vitro or in vivo, e.g., shRNA, from a DNA template. The dsRNA molecules can be designed using any method known in the art. Negative control siRNAs should not have significant sequence complementarity to the appropriate genome. Such negative controls can be designed by randomly scrambling the nucleotide sequence of the selected siRNA; a homology search can be performed to ensure that the negative control lacks homology to any other gene in the appropriate genome. In addition, negative control siRNAs can be designed by introducing one or more base mismatches into the sequence.

The methods described herein can use both siRNA and modified siRNA derivatives, e.g., siRNAs modified to alter a property such as the specificity and/or pharmacokinetics of the composition, for example, to increase half-life in the body, e.g., crosslinked siRNAs. Thus, the invention includes methods of administering siRNA derivatives that include siRNA having two complementary strands of nucleic acid, such that the two strands are crosslinked. The oligonucleotide modifications include, but not limited to, 2′-O-methyl, 2′-fluoro, 2′-O-methyoxyethyl and phosphorothiate, boranophosphate, 4′-thioribose. (Wilson and Keefe, Curr. Opin. Chem. Biol. 10:607-614 (2006); Prakash et al., J. Med. Chem. 48:4247-4253 (2005); Soutschek et al., Nature 432:173-178 (2004)).

In some embodiments, the siRNA derivative has at its 3′ terminus a biotin molecule (e.g., a photocleavable biotin), a peptide (e.g., a Tat peptide), a nanoparticle, a peptidomimetic, organic compounds (e.g., a dye such as a fluorescent dye), or dendrimer. Modifying siRNA derivatives in this way may improve cellular uptake or enhance cellular targeting activities of the resulting siRNA derivative as compared to the corresponding siRNA, are useful for tracing the siRNA derivative in the cell, or improve the stability of the siRNA derivative compared to the corresponding siRNA.

The inhibitory nucleic acid compositions can be unconjugated or can be conjugated to another moiety, such as a nanoparticle, to enhance a property of the compositions, e.g., a pharmacokinetic parameter such as absorption, efficacy, bioavailability, and/or half-life. The conjugation can be accomplished by methods known in the art, e.g., using the methods of Lambert et al., Drug Deliv. Rev.:47(1), 99-112 (2001) (describes nucleic acids loaded to polyalkylcyanoacrylate (PACA) nanoparticles); Fattal et al., J. Control Release 53(1-3):137-43 (1998) (describes nucleic acids bound to nanoparticles); Schwab et al., Ann. Oncol. 5 Suppl. 4:55-8 (1994) (describes nucleic acids linked to intercalating agents, hydrophobic groups, polycations or PACA nanoparticles); and Godard et al., Eur. J. Biochem. 232(2):404-10 (1995) (describes nucleic acids linked to nanoparticles). The inhibitory nucleic acid molecules can also be labeled using any method known in the art; for instance, the nucleic acid compositions can be labeled with a fluorophore, e.g., Cy3, fluorescein, or rhodamine. The labeling can be carried out using a kit, e.g., the SILENCER™ siRNA labeling kit (Ambion). Additionally, the siRNA can be radiolabeled, e.g., using 3H, 32P, or other appropriate isotope.

siRNA Delivery

Direct delivery of siRNA in saline or other excipients can silence target genes in tissues, such as the eye, lung, and central nervous system (Bitko et al., Nat. Med. 11:50-55 (2005); Shen et al., Gene Ther. 13:225-234 (2006); Thakker, et al., Proc. Natl. Acad. Sci. U.S.A. (2004)). In adult mice, efficient delivery of siRNA can be accomplished by “high-pressure” delivery technique, a rapid injection (within 5 seconds) of a large volume of siRNA containing solution into animal via the tail vein (Liu (1999), supra; McCaffrey (2002), supra; Lewis, Nature Genetics 32:107-108 (2002)).

Liposomes and nanoparticles can also be used to deliver siRNA into animals. Delivery methods using liposomes, e.g. stable nucleic acid-lipid particles (SNALPs), dioleoyl phosphatidylcholine (DOPC)-based delivery system, as well as lipoplexes, e.g., Lipofectamine 2000, TransIT-TKO, have been shown to effectively repress target mRNA (de Fougerolles, Human Gene Ther. 19:125-132 (2008); Landen et al., Cancer Res. 65:6910-6918 (2005); Luo et al., Mol Pain 1:29 (2005); Zimmermann et al., Nature 441:111-114 (2006)). Conjugating siRNA to peptides, RNA aptamers, antibodies, or polymers, e.g. dynamic polyconjugates, cyclodextrin-based nanoparticles, atelocollagen, and chitosan, can improve siRNA stability and/or uptake (Howard et al., Mol. Ther. 14:476-484 (2006); Hu-Lieskovan et al., Cancer Res. 65:8984-8992 (2005); Kumar, et al., Nature 448:39-43; McNamara et al., Nat. Biotechnol. 24:1005-1015 (2007); Rozema et al., Proc. Natl. Acad. Sci. U.S.A. 104:12982-12987 (2007); Song et al., Nat. Biotechnol. 23:709-717 (2005); Soutschek (2004), supra; Wolfrum et al., Nat. Biotechnol. 25:1149-1157 (2007)).

Viral-mediated delivery mechanisms can also be used to induce specific silencing of targeted genes through expression of siRNA, for example, by generating recombinant adenoviruses harboring siRNA under RNA Pol II promoter transcription control (Xia et al. (2002), supra). Infection of HeLa cells by these recombinant adenoviruses allows for diminished endogenous target gene expression. Injection of the recombinant adenovirus vectors into transgenic mice expressing the target genes of the siRNA results in in vivo reduction of target gene expression. Id. In an animal model, whole-embryo electroporation can efficiently deliver synthetic siRNA into post-implantation mouse embryos (Calegari et al., Proc. Natl. Acad. Sci. USA 99(22):14236-40 (2002)).

Stable siRNA Expression

Synthetic siRNAs can be delivered into cells, e.g., by direct delivery, cationic liposome transfection, and electroporation. However, these exogenous siRNA typically only show short term persistence of the silencing effect (4-5 days). Several strategies for expressing siRNA duplexes within cells from recombinant DNA constructs allow longer-term target gene suppression in cells, including mammalian Pol II and III promoter systems (e.g., H1, U1, or U6/snRNA promoter systems (Denti et al. (2004), supra; Tuschl (2002), supra); capable of expressing functional double-stranded siRNAs (Bagella et al., J. Cell. Physiol. 177:206-213 (1998); Lee et al. (2002), supra; Miyagishi et al. (2002), supra; Paul et al. (2002), supra; Scherer et al. (2007), supra; Yu et al. (2002), supra; Sui et al. (2002), supra).

Transcriptional termination by RNA Pol III occurs at runs of four consecutive T residues in the DNA template, providing a mechanism to end the siRNA transcript at a specific sequence. The siRNA is complementary to the sequence of the target gene in 5′-3′ and 3′-5′ orientations, and the two strands of the siRNA can be expressed in the same construct or in separate constructs. Hairpin siRNAs, driven by H1 or U6 snRNA promoter and expressed in cells, can inhibit target gene expression (Bagella et al. (1998), supra; Lee et al. (2002), supra; Miyagishi et al. (2002), supra; Paul et al. (2002), supra; Yu et al. (2002), supra; Sui et al. (2002) supra). Constructs containing siRNA sequence under the control of T7 promoter also make functional siRNAs when cotransfected into the cells with a vector expression T7 RNA polymerase (Jacque (2002), supra).

In another embodiment, siRNAs can be expressed in a miRNA backbone which can be transcribed by either RNA Pol II or III. MicroRNAs are endogenous noncoding RNAs of approximately 22 nucleotides in animals and plants that can post-transcriptionally regulate gene expression (Bartel, Cell 116:281-297 (2004); Valencia-Sanchez et al, Genes & Dev. 20:515-524 (2006)) One common feature of miRNAs is that they are excised from an approximately 70 nucleotide precursor RNA stem loop by Dicer, an RNase III enzyme, or a homolog thereof. By substituting the stem sequences of the miRNA precursor with the sequence complementary to the target mRNA, a vector construct can be designed to produce siRNAs to initiate RNAi against specific mRNA targets in mammalian cells. When expressed by DNA vectors containing polymerase II or III promoters, miRNA designed hairpins can silence gene expression (McManus (2002), supra; Zeng (2002), supra).

Uses of Engineered RNA Precursors to Induce RNAi

Engineered RNA precursors, introduced into cells or whole organisms as described herein, will lead to the production of a desired siRNA molecule. Such an siRNA molecule will then associate with endogenous protein components of the RNAi pathway to bind to and target a specific mRNA sequence for cleavage, destabilization, and/or translation inhibition destruction. In this fashion, the mRNA to be targeted by the siRNA generated from the engineered RNA precursor will be depleted from the cell or organism, leading to a decrease in the concentration of the protein encoded by that mRNA in the cell or organism.

Antisense

An “antisense” nucleic acid can include a nucleotide sequence that is complementary to a “sense” nucleic acid encoding a protein, e.g., complementary to the coding strand of a double-stranded cDNA molecule or complementary to a target mRNA sequence. The antisense nucleic acid can be complementary to an entire coding strand of a target sequence, or to only a portion thereof (for example, the coding region of a target gene). In another embodiment, the antisense nucleic acid molecule is antisense to a “noncoding region” of the coding strand of a nucleotide sequence encoding the selected target gene (e.g., the 5′ and 3′ untranslated regions).

An antisense nucleic acid can be designed such that it is complementary to the entire coding region of a target mRNA but can also be an oligonucleotide that is antisense to only a portion of the coding or noncoding region of the target mRNA. For example, the antisense oligonucleotide can be complementary to the region surrounding the translation start site of the target mRNA, e.g., between the −10 and +10 regions of the target gene nucleotide sequence of interest. An antisense oligonucleotide can be, for example, about 7, 10, 15, 20, 25, 30, 35, 40, 45, 50, 55, 60, 65, 70, 75, 80, or more nucleotides in length.

An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used.

The antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

Based upon the sequences disclosed herein, one of skill in the art can easily choose and synthesize any of a number of appropriate antisense molecules for use in accordance with the present invention. For example, a “gene walk” comprising a series of oligonucleotides of 15-30 nucleotides spanning the length of a target nucleic acid can be prepared, followed by testing for inhibition of target gene expression. Optionally, gaps of 5-10 nucleotides can be left between the oligonucleotides to reduce the number of oligonucleotides synthesized and tested.

The antisense nucleic acid molecules of the invention are typically administered to a subject (e.g., by direct injection at a tissue site), or generated in situ such that they hybridize with or bind to cellular mRNA and/or genomic DNA encoding a target protein to thereby inhibit expression of the protein, e.g., by inhibiting transcription, splicing, and/or translation. Alternatively, antisense nucleic acid molecules can be modified to target selected cells and then administered systemically. For systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter can be used.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al., Nucleic Acids. Res. 15:6625-6641 (1987)). The antisense nucleic acid molecule can also comprise a 2′-O-methylribonucleotide (Inoue et al. Nucleic Acids Res. 15:6131-6148 (1987)), 2′-O-methoxyethylribonucleotide, locked nucleic acid, ethylene-bridged nucleic acid, oxetane-modified ribose, peptide nucleic acid, or a chimeric RNA-DNA analogue (Inoue et al. FEBS Lett., 215:327-330 (1987)).

In some embodiments, the antisense nucleic acid is a morpholino oligonucleotide (see, e.g., Heasman, Dev. Biol. 243:209-14 (2002); Iversen, Curr Opin. Mol. Ther. 3:235-8 (2001); Summerton, Biochim. Biophys. Acta. 1489:141-58 (1999).

Target gene expression can be inhibited by targeting nucleotide sequences complementary to a regulatory region, e.g., promoters and/or enhancers) to form triple helical structures that prevent transcription of the target gene in target cells. See generally, Helene, C. Anticancer Drug Des. 6:569-84 (1991); Helene, C. Ann. N.Y. Acad. Sci. 660:27-36 (1992); and Maher, Bioassays 14:807-15 (1992). The potential sequences that can be targeted for triple helix formation can be increased by creating a so called “switchback” nucleic acid molecule. Switchback molecules are synthesized in an alternating 5′-3′,3′-5′ manner, such that they base pair with first one strand of a duplex and then the other, eliminating the necessity for a sizeable stretch of either purines or pyrimidines to be present on one strand of a duplex.

Ribozymes

Ribozymes are a type of RNA that can be engineered to enzymatically cleave and inactivate other RNA targets in a specific, sequence-dependent fashion. By cleaving the target RNA, ribozymes inhibit translation, thus preventing the expression of the target gene. Ribozymes can be chemically synthesized in the laboratory and structurally modified to increase their stability and catalytic activity using methods known in the art. Alternatively, ribozyme genes can be introduced into cells through gene-delivery mechanisms known in the art. A ribozyme having specificity for a target-protein encoding nucleic acid can include one or more sequences complementary to the nucleotide sequence of a target cDNA disclosed herein, and a sequence having known catalytic sequence responsible for mRNA cleavage (see U.S. Pat. No. 5,093,246 or Haselhoff and Gerlach Nature 334:585-591 (1988)). For example, a derivative of a Tetrahymena L-19 IVS RNA can be constructed in which the nucleotide sequence of the active site is complementary to the nucleotide sequence to be cleaved in a target mRNA. See, e.g., Cech et al. U.S. Pat. No. 4,987,071; and Cech et al. U.S. Pat. No. 5,116,742. Alternatively, a target mRNA can be used to select a catalytic RNA having a specific ribonuclease activity from a pool of RNA molecules. See, e.g., Bartel and Szostak, Science 261:1411-1418 (1993).

Methods for Identifying Compounds that Increase or Decrease Activity or Level of AATF, HRD1, or ATF6

The invention also provides screening methods (also referred to herein as “screening assays”) for identifying compounds (e.g., peptides, peptidomimetics, small molecules, or other compounds) that increase or decrease AATF, HRD1, or ATF6 level or activities, by e.g., increasing or decreasing expression of AATF, HRD1, or ATF6 or by enhancing or inhibiting AATF, HRD1, or ATF6's activity. Such compounds can be further tested to determine whether they decrease ER stress signaling or inhibit ER-stress induced cell death in vivo, e.g., an animal, or in vitro, e.g., in cultured cells.

Libraries of Test Compounds

In certain embodiments, screens disclosed herein utilize libraries of test compounds. As used herein, a “test compound” can be any chemical compound, for example, a macromolecule (e.g., a polypeptide, a protein complex, glycoprotein, polysaccharide, or a nucleic acid) or a small molecule (e.g., an amino acid, a nucleotide, or an organic or inorganic compound). A test compound can have a formula weight of less than about 10,000 grams per mole, less than 5,000 grams per mole, less than 1,000 grams per mole, or less than about 500 grams per mole. The test compound can be naturally occurring (e.g., an herb or a natural product), synthetic, or can include both natural and synthetic components. Examples of test compounds include peptides, peptidomimetics (e.g., peptoids, retro-peptides, inverso peptides, and retro-inverso peptides), amino acids, amino acid analogs, polynucleotides, polynucleotide analogs, nucleotides, nucleotide analogs, and organic or inorganic compounds, e.g., heteroorganic or organometallic compounds.

Test compounds can be screened individually or in parallel. An example of parallel screening is a high throughput drug screen of large libraries of chemicals. Such libraries of candidate compounds can be generated or purchased, e.g., from Chembridge Corp., San Diego, Calif. Libraries can be designed to cover a diverse range of compounds. For example, a library can include 500, 1000, 10,000, 50,000, or 100,000 or more unique compounds. Alternatively, prior experimentation and anecdotal evidence can suggest a class or category of compounds of enhanced potential. A library can be designed and synthesized to cover such a class of chemicals.

The synthesis of combinatorial libraries is well known in the art and has been reviewed (see, e.g., Gordon et al., J. Med. Chem., 37:1385-1401 (1994); Hobbes et al, Acc. Chem. Res., 29:114 (1996); Armstrong, et al., Acc. Chem. Res., (1996) 29:123; Ellman, Acc. Chem. Res., (1996) 29:132; Gordon et al., Acc. Chem. Res., 29:144 (1996); Lowe, Chem. Soc. Rev., 309 (1995); Blondelle et al., Trends Anal. Chem., 14:83 (1995); Chen et al., J. Am. Chem. Soc., 116:2661 (1994); U.S. Pat. Nos. 5,359,115, 5,362,899, and 5,288,514; PCT Publication Nos. WO92/10092, WO93/09668, WO91/07087, WO93/20242, and WO94/08051).

Libraries of compounds can be prepared according to a variety of methods, some of which are known in the art. For example, a “split-pool” strategy can be implemented in the following way: beads of a functionalized polymeric support are placed in a plurality of reaction vessels; a variety of polymeric supports suitable for solid-phase peptide synthesis are known, and some are commercially available (for examples, see, e.g., M. Bodansky “Principles of Peptide Synthesis,” 2nd edition, Springer-Verlag, Berlin (1993)). To each aliquot of beads is added a solution of a different activated amino acid, and the reactions are allowed to proceed to yield a plurality of immobilized amino acids, one in each reaction vessel. The aliquots of derivatized beads are then washed, “pooled” (i.e., recombined), and the pool of beads is again divided, with each aliquot being placed in a separate reaction vessel. Another activated amino acid is then added to each aliquot of beads. The cycle of synthesis is repeated until a desired peptide length is obtained. The amino acid residues added at each synthesis cycle can be randomly selected; alternatively, amino acids can be selected to provide a “biased” library, e.g., a library in which certain portions of the inhibitor are selected non-randomly, e.g., to provide an inhibitor having known structural similarity or homology to a known peptide capable of interacting with an antibody, e.g., the an anti-idiotypic antibody antigen binding site. It will be appreciated that a wide variety of peptidic, peptidomimetic, or non-peptidic compounds can be readily generated in this way.

The “split-pool” strategy can result in a library of peptides, e.g., modulators, which can be used to prepare a library of test compounds of the invention. In another illustrative synthesis, a “diversomer library” is created by the method of Hobbs DeWitt et al. (Proc. Natl. Acad. Sci. U.S.A., 90:6909 (1993)). Other synthesis methods, including the “tea-bag” technique of Houghten (see, e.g., Houghten et al., Nature, 354:84-86 (1991)) can also be used to synthesize libraries of compounds according to the subject invention.

Libraries of compounds can be screened to determine whether any members of the library can increase or decrease AATF, HRD1, or ATF6 expression level or activity and, if so, to identify the activating or deactivating compound. Methods of screening combinatorial libraries have been described (see, e.g., Gordon et al., J. Med. Chem., supra). Exemplary assays useful for screening libraries of test compounds are described above.

Screens

Screens for compounds for treating ER stress disorders can be performed by identifying from a group of test compounds those that, e.g., increase AATF or HRD1 expression level or activity or decrease ATF6 expression level or activity. Such compounds are candidate compounds that activate AATF or HRD1 or deactivate ATF6, and such compounds can be further tested for their ability to decrease ER stress signaling in vitro or in vivo. Such compounds can also be further tested for their ability to increase Akt1 expression level or phosphorylation in vivo or in vitro. Such compounds can also be tested for their ability to inhibit ER-stress related (e.g., caused or induced) cell death in vivo or in vitro. Such compounds are candidate compounds that treat ER stress disorders, and such candidate compounds can be further assayed for their ability to treat ER stress disorders in animal models.

The screens described herein can be performed by providing a model system, e.g., a cell or an animal, contacting the model system with a test compound, and comparing the expression level or activity of AATF, HRD1, or ATF6 in the model system in the presence and in the absence of the test compound. If AATF or HRD1 level or activity is increased in the presence of a compound, the compound is a candidate activator. If ATF6 level or activity is decreased in the presence of a compound, the compound is a candidate deactivator. Candidate compounds can be further tested for their ability to decrease ER stress signaling in vivo or in vitro using methods described herein. Candidate compounds can also be further tested for their ability to inhibit cell death, e.g., apoptosis, associated with (e.g., induced by, caused by) ER stress in vivo or in vitro as described herein, e.g., using TUNEL assays. Such candidate compounds can be further assayed for their ability to treat ER stress disorders in animal models.

In some embodiments, candidate compounds that increase AATF or HRD1 or decrease ATF6 level or activity are further tested for their ability to increase the level of Akt1 expression or phosphorylation. Conventional methods known in the art can be used to assay the level of Akt1 expression or phosphorylation, e.g., using anti-Akt1 antibodies. For example, reporter constructs, in which the promoter region of the Akt1 gene is operably linked to a reporter gene (e.g., luciferase gene) as described herein, can be used to measure the ability of candidate compounds to increase Akt1 expression. Other methods can be used to measure Akt1 expression.

In some embodiments, ER stress level is induced in the model system before contacting the model system with a test compound. Methods are known in the art for inducing ER stress. For example, ER stress can be induced in a model system, e.g., an animal or a cell, by administering a compound known to cause ER dysfunction, e.g., by administering a sublethal dose of thapsigargin, tunicamycin (e.g., 0.25-1 mg/kg tunicamycin; see Zinszner et al., Genes and Dev. 12:982-995 (1998)), or a proteosome inhibitor, e.g., lactacystin. Other methods can be used to induce ER stress in a model system.

Model systems suitable for the screening methods described herein include cells, e.g., pancreatic β-cells (e.g., MIN6 cells), rat insulinoma cells, COS7 cells, Neuro2a cells, dopamine producing neurons, and human neuroblastoma cells. Model systems can also include ER stress disorder animal models, e.g., the Akita mouse model for diabetes. Skilled practitioners would readily appreciate that a number of cells or animal models could be used in the screening methods described herein, and that which model system to be used depends on the compounds to be identified, e.g., which ER stress disorder is to be treated by the compound. In some instances, the model system is a model of a neurodegenerative disease. In other instances, the model system is a model of diabetes. Assays disclosed herein may be carried out in whole cell preparations and/or in ex vivo cell-free systems.

Medicinal Chemistry

Once a compound (or agent) of interest has been identified, standard principles of medicinal chemistry can be used to produce derivatives of the compound. Derivatives can be screened for improved pharmacological properties, for example, efficacy, pharmaco-kinetics, stability, solubility, and clearance. The moieties responsible for a compound's activity in the assays described above can be delineated by examination of structure-activity relationships (SAR) as is commonly practiced in the art. A person of ordinary skill in pharmaceutical chemistry could modify moieties on a candidate compound or agent and measure the effects of the modification on the efficacy of the compound or agent to thereby produce derivatives with increased potency. For an example, see Nagarajan et al., J. Antibiot. 41:1430-8 (1988). Furthermore, if the biochemical target of the compound (or agent) is known or determined, the structure of the target and the compound can inform the design and optimization of derivatives. Molecular modeling software is commercially available (e.g., Molecular Simulations, Inc.) for this purpose.

III. Methods for Screening ER Stress Signaling Modulators

Evidence provided herein shows that Wolfram syndrome 1 (WFS1) and Activating Transcription Factor 6 (ATF6) form a complex with the proteasome and an E3 ligase, hydroxymethylglutaryl reductase degradation 1 (HRD1), on the ER membrane, leading to degradation of ATF6 under non-stress conditions. Accordingly, the invention provides methods for identifying compounds that can modulate, e.g., increase or decrease, ER stress signaling by screening for compounds that modulate, e.g., increase or decrease, the protein-protein interactions between WFS1, ATF6, and HRD1, e.g., between WFS1 and ATF6, between WFS1 and HRD1, and between ATF6 and HRD1. Test compounds that can modulate protein-protein interactions are candidate compounds for modulating ER stress signaling. Such candidate compounds can be further tested for their ability to modulate ATF6 protein level. Candidate compounds that increase ATF6 protein level are compounds that are expected to increase ER stress signaling. Such compounds can be used, e.g., to induce ER stress in a model system. Candidate compounds that decrease ATF6 protein level are compounds that are expected to decrease ER stress signaling. Such compounds can be tested for their ability to decrease ER stress signaling in vivo or in vitro. Such candidate compounds can be further tested for their ability to inhibit ER-stress induced cell death in vivo or in vitro. Candidate compounds can also be further tested for their ability to treat ER stress disorders in animal models.

Nucleic Acid and Polypeptide

WFS1, ATF6, and HRD1 polypeptides or biologically active fragments thereof, and nucleic acids encoding full-length WFS1, ATF6, or HRD1 polypeptides or biologically active fragments thereof are useful for the screening methods described herein. WFS1, ATF6, and HRD1 polypeptides and nucleic acids encoding them are readily obtained by one of ordinary skill in the art without undue experimentation. For example, the amino acid and nucleic acid sequences of human WFS1 are known (see, e.g., GenBank Ace. No. AF084481.1 for a nucleic acid sequence and GenBank Ace. No. O76024.1 for an amino acid sequence). Human HRD1 amino acid and nucleic acid sequences are also known (e.g., Genbank Ace. No. NP_{—}115807.1 or NP_{—}757385.1). Further, human ATF6 amino acid and nucleic acid sequences are known (see, e.g., Genbank Ace. No. AB015856.1 or P18850.3). Anucleic acid encoding a mammalian, e.g., human, WFS1, ATF6 or HRD1 amino acid sequences can be amplified from human cDNA by conventional PCR techniques, using primers upstream and downstream of the coding sequence. WFS1, ATF6 and HRD1 polypeptides or fragments thereof can be produced and isolated using methods described herein.

Screens

Screens for compounds that modulate ER stress signaling can be performed by identifying from a group of test compounds those that modulate protein-protein interactions between WFS1, ATF6 and HRD1 polypeptides or fragments thereof, e.g., between WFS1 and ATF6, between WFS1 and HRD1, between ATF6 and HRD1, or between WFS1, ATF6 and HRD1. Such candidate compounds can be further tested for their ability to modulate ATF6 levels or activity in a model system, e.g., a cell or an animal. Such compounds are candidate compounds that modulate ER stress signaling, e.g., increase or decrease ER stress signaling.

Screens for compounds for treating ER stress disorders can be performed by identifying from a group of test compounds those that, e.g., increase WFS1 protein-protein interactions with an ATF6 and/or HRD1 polypeptide or a biologically active fragment thereof, and/or increase ATF6 protein-protein interactions with an WFS1 and/or HRD1 polypeptide or a biologically active fragment thereof. Such compounds are candidate compounds that reduce ER stress signaling. These candidate compounds can be further tested for their ability to decrease ATF6 level, e.g., by increasing ATF6 ubiquitination or protein degradation, and such compounds can be further tested for their ability to inhibit ER-stress induced cell death. Such compounds are candidate compounds that treat ER stress disorders, and such candidate compounds can be further assayed for their ability to treat ER stress disorders in animal models.

Test compounds that modulate interactions between WFS1, ATF6, and HRD1 polypeptides or biologically active fragments thereof, e.g., between WFS1 and ATF6, between WFS1 and HRD1, between ATF6 and HRD1, or between WFS1, ATF6, and HRD1, are referred to herein as “candidate compounds.” Assays disclosed herein may be carried out in whole cell preparations and/or in ex vivo cell-free systems.

A method useful for high throughput screening of compounds capable of modulating protein-protein interactions is described in Lepourcelet et al., Cancer Cell 5: 91-102 (2004), which is incorporated herein by reference in its entirety. Typically, a first protein is provided. The first protein is an WFS1, ATF6 or HRD1 polypeptide, or a biologically active fragment thereof. A second protein is provided, which is different from the first protein and which is labeled. The second protein is an WFS1, ATF6 or HRD1 polypeptide, or a biologically active fragment thereof. A test compound is provided. The first protein, second protein, and test compound are contacted with each other. The amount of label bound to the first protein is then determined. A change in protein-protein interaction (e.g., binding) between the first protein and the second protein as assessed by the amount of label bound is indicative of the usefulness of the compound in modulating protein-protein interactions between the first and second polypeptides. In some embodiments, the change is assessed relative to the same reaction without addition of the test compound.

In certain embodiments, the first protein is attached to a solid support. Solid supports include, e.g., resins such as agarose, beads, and multiwell plates. In certain embodiments, the method includes a washing step after the contacting step, so as to separate bound and unbound label.

In certain embodiments, a plurality of test compounds is contacted with the first protein and the second protein. The different test compounds can be contacted with the other compounds in groups or separately. In certain embodiments, each of the test compounds is contacted with both the first protein and the second protein in separate wells. For example, the method can be used to screen libraries of test compounds, discussed in detail above. Libraries can include, e.g., natural products, organic chemicals, peptides, and/or modified peptides, including, e.g., D-amino acids, unconventional amino acids, and N-substituted amino acids. Typically, the libraries are in a form compatible with screening in multiwell plates, e.g., 96-well plates. The assay is particularly useful for automated execution in a multiwell format in which many of the steps are controlled by computer and carried out by robotic equipment. The libraries can also be used in other formats, e.g., synthetic chemical libraries affixed to a solid support and available for release into microdroplets.

In certain embodiments, the first protein is a WFS1 polypeptide, or a biologically active fragment thereof, and the second protein is an ATF6 polypeptide, or a biologically active fragment thereof. In other embodiments, the first protein is a WFS1 polypeptide, or a biologically active fragment thereof, and the second protein is a HRD1 polypeptide, or a biologically active fragment thereof. In other embodiments, the first protein is an ATF6 polypeptide, or a biologically fragment thereof, and the second protein is a HRD1 polypeptide, or a biologically fragment thereof. The solid support to which the first protein is attached can be, e.g., SEPHAROSE™ beads, scintillation proximity assay (SPA) beads (microspheres that incorporate a scintillant) or a multiwell plate. SPA beads can be used when the assay is performed without a washing step, e.g., in a scintillation proximity assay. SEPHAROSE™ beads can be used when the assay is performed with a washing step. The second protein can be labeled with any label that will allow its detection, e.g., a radiolabel, a fluorescent agent, biotin, a peptide tag, or an enzyme fragment. The second protein can also be radiolabeled, e.g., with ¹²⁵I or ³H.

In certain embodiments, the enzymatic activity of an enzyme chemically conjugated to, or expressed as a fusion protein with, the first or second protein, is used to detect bound protein. A binding assay in which a standard immunological method is used to detect bound protein is also included.

In certain other embodiments, the interaction of a first protein and a second protein is detected by fluorescence resonance energy transfer (FRET) between a donor fluorophore covalently linked to a first protein (e.g., a fluorescent group chemically conjugated to a peptide disclosed herein, or a variant of green fluorescent protein (GFP) expressed as a GFP chimeric protein linked to a peptide disclosed herein) and an acceptor fluorophore covalently linked to a second protein, where there is suitable overlap of the donor emission spectrum and the acceptor excitation spectrum to give efficient nonradiative energy transfer when the fluorophores are brought into close proximity through the protein-protein interaction of the first and second protein. Alternatively, both the donor and acceptor fluorophore can be conjugated at each end of the same peptide, e.g., a WFS1 polypeptide. The free peptide has high FRET efficiency due to intramolecular FRET between donor and acceptor sites causing quenching of fluorescence intensity. Upon binding to, e.g., ATF6, the intramolecular FRET of the peptide-dye conjugate decreases, and the donor signal increases. In another embodiment, fluorescence polarization (FP) is used to monitor the interaction between two proteins. For example, a fluorescently labeled peptide will rotate at a fast rate and exhibit low fluorescence polarization. When bound to a protein, the complex rotates more slowly, and fluorescence polarization increases.

In other embodiments, the protein-protein interaction is detected by reconstituting domains of an enzyme, e.g., beta-galactosidase (see Rossi et al, Proc. Natl. Acad. Sci. USA, 94:8405-8410 (1997)).

In still other embodiments, the protein-protein interaction is assessed by fluorescence ratio imaging (Bacskai et al, Science, 260:222-226 (1993)) of suitable chimeric constructs of a first and second protein, or by variants of the two-hybrid assay (Fearon et al., Proc. Nat'l. Acad. Sci. USA, 89:7958-7962 (1992); Takacs et al., Proc. Natl. Acad. Sci. USA, 90:10375-10379 (1993); Vidal et al., Proc. Nat.'l. Acad. Sci. USA, 93:10315-10320 (1996); Vidal et al, Proc. Nat'l Acad. Sci. USA, 93:10321-10326 (1996)) employing suitable constructs of first and second protein tailored for a high throughput assay to detect compounds that inhibit the first protein/second protein interaction. These embodiments have the advantage that the cell permeability of compounds that act as modulators in the assay is assured.

For example, in one assay, but not the only assay, e.g., a WFS1, ATF6, or HRD1 polypeptide, or a biologically active fragment thereof is adsorbed to ELISA plates. The adsorbed polypeptides are then exposed to test compounds, followed by exposure to e.g., a WFS1, ATF6, or HRD1 polypeptide, or a biologically active fragment thereof (optionally fused to a reporter peptide such as Glutathione S-transferase). ELISA plates are washed and bound protein is detected using, e.g., anti-WFS1, anti-ATF6, or anti-HRD1 antibodies (or an antibody that selectively binds the reporter peptide). The antibody can be detected either directly or indirectly using a secondary antibody. Compounds that interfere with protein-protein interactions yield reduced antibody signal in the ELISA plates.

In some embodiments, candidate compounds that can modulate ER stress signaling can be identified by providing a model system, e.g., a cell or an animal, contacting the model system with a test compound, and comparing the level of a protein complex comprising WFS1, ATF6, and HRD1 in the model system in the presence and in the absence of the test compound, such that a different level of the protein complex in the presence of the test compound than in its absence indicates that the test compound is a candidate compound for modulating ER stress signaling. The level of a protein complex can be assayed using conventional methods, e.g., immunoprecipitation and immunoblotting.

Candidate compounds can be further tested for their ability to modulate ER stress signaling as described above. Candidate compounds can also be further tested for their ability to modulate ATF6 activities (e.g., its ability to modulate transcription of ATF6 target genes), level of ATF6 protein, or level of ubiquitinated ATF6 protein in cells. Levels of ATF6 protein and level of ubiquitinated ATF6 protein can be assayed by methods well known in the art, e.g., immunoblotting. Level of ATF6 activity can be assayed, e.g., using an ATF6 binding site reporter gene as described in Example 1 herein. Candidate compounds can be further tested for their ability to inhibit ER stress induced cell death in vivo, e.g., in an animal model, or in vitro, e.g., in cultured cells, using methods described herein and other methods known in the art.

Candidate compounds can be retested, e.g. on $-cells, e.g., in vitro, or tested on animals, e.g., animals that are models for ER stress disorder. Candidate compounds that are positive in a retest can be considered “lead” compounds to be further optimized and derivatized, or may be useful therapeutic or diagnostic compounds themselves.

IV. Methods of Making Insulin-Producing Cells and Therapeutic Methods Using the Same

Evidence described below demonstrates that up-regulating the expression of WFS1 in exocrine pancreatic cells, which do not express WFS1 or produce insulin endogenously, induces insulin production in these cells. Accordingly, provided herein are exocrine pancreatic cells, e.g., acinar cells, that produce insulin and methods for treating diabetes in a patient by, e.g., increasing WFS1 expression in the exocrine pancreatic cells in the patient, or by administering to the patient exocrine pancreatic cells expressing WFS1.

The invention includes exocrine pancreatic cells engineered or treated to produce insulin, e.g., by up-regulating the expression of WFS1. Methods using known techniques can be used to up-regulate the expression of WFS1 in exocrine pancreatic cells. For example, exocrine pancreatic cells can be transfected with an inducible lentivirus expressing human WFS1 as described herein.

Also provided herein are methods for treating diabetes in a patient by up-regulating the expression of WFS1 in the exocrine pancreatic cells in the patient, e.g., by administering nucleic acid molecules encoding WFS1 polypeptides. Methods describe herein can be used for administering genetic constructs (e.g., vectors and plasmids) that include a WFS1 nucleic acid described herein, operably linked to a transcription and/or translation sequence to enable expression, e.g., expression vectors. In some instances, the expression vectors can be administered into the pancreas of the patient, by e.g., direct injection of the vectors into the pancreas.

Compounds that up-regulate the expression or activity of WFS1 in exocrine pancreatic cells in the patient can also be used. For example, evidence suggests that valproic acid, a compound used to treat epilepsy, bipolar disorder, and clinical depression, can increase WFS1 expression or activity. Valproic acid can be administered locally into the pancreas of a patient with diabetes to specifically increase WFS1 expression in cells of the pancreas, thereby inducing exocrine pancreatic cells to produce insulin. Compounds that increase WFS1 expression in exocrine pancreatic cells can also be identified by screening libraries of test compounds. An exemplary screening method can include providing an exocrine pancreatic cell, contacting the cell with a test compound, and comparing the expression level of WFS1 in the presence and in the absence of the test compound. Candidate compounds that increase WFS1 expression level can be further tested for their ability to induce insulin productions in cells that do not normally produce insulin, e.g., exocrine pancreatic cells. Such candidate compounds are candidate compounds for treating diabetes.

The invention also provides methods for treating diabetes in a patient by administering to the patient exocrine pancreatic cells that produce insulin. The insulin-producing exocrine pancreatic cells can be generated as described herein. In some instances, the insulin-producing exocrine pancreatic cells are derived from the patient to be treated. For example, conventional methods can be used to harvest exocrine pancreatic cells from the patient, and then the cells can be engineered or treated to express WFS1 and produce insulin using methods described herein.

Methods known in the art can be used to administer the insulin-producing exocrine pancreatic cells to a patient, e.g., using a delivery system configured to allow the introduction of cells into a subject. In general, the delivery system can include a reservoir containing a population of cells including insulin-producing exocrine pancreatic cells, and a needle in fluid communication with the reservoir. Typically, the population of insulin-producing exocrine pancreatic cells will be in a pharmaceutically acceptable carrier, with or without a scaffold, matrix, or other implantable device to which the cells can attach (examples include carriers made of, e.g., collagen, fibronectin, elastin, cellulose acetate, cellulose nitrate, polysaccharide, fibrin, gelatin, and combinations thereof). Such delivery systems are also within the scope of the invention. Generally, such delivery systems are maintained in a sterile manner. Various routes of administration and various sites (e.g., renal sub capsular, subcutaneous, central nervous system (including intrathecal), intravascular, intrahepatic, intrasplanchnic, intraperitoneal (including intraomental), intramuscularly implantation) can be used. Generally, the cells will be implanted into the subject subcutaneously. In some embodiments, the population of insulin-producing exocrine pancreatic cells that is implanted includes at least 10⁷, 10⁸, 10⁹, or more cells.

Where non-immunologically compatible cells are used, e.g., cells from a source other than the patient to be treated, an immunosuppressive compound, e.g., a drug or antibody, can be administered to the recipient subject at a dosage sufficient to achieve inhibition of rejection of the cells. Dosage ranges for immunosuppressive drugs are known in the art. See, e.g., Freed et al., N. Engl. J. Med. 327:1549 (1992); Spencer et al., N. Engl. J. Med. 327:1541 (1992); Widner et al., N. Engl. J. Med. 327:1556 (1992)). Dosage values may vary according to factors such as the disease state, age, sex, and weight of the individual.

V. Kits for Screening for ER Stress Signaling Modulators

Provided herein are kits for identifying compounds that modulate ER stress signaling by, e.g., modulating the protein-protein interaction between WFS1, ATF6 and HRD1, using, for example, the screening assays described herein. Various combinations of WFS1, ATF6 and HRD1 polypeptides, e.g., WFS1 and ATF6 polypeptides, WFS1 and HRD1 polypeptides, ATF6 and HRD1 polypeptides, or all three, can be provided in a kit. The kit can include, for example, WFS1 polypeptides or fragments thereof as described above, and ATF6 polypeptides or fragments thereof as described above. In some embodiments, the kit can include, for example, WFS1 polypeptides or fragments thereof as described above, and HRD1 polypeptides or fragments thereof as described above. In other embodiments, the kit can include, for example, ATF6 polypeptides or fragments thereof as described above, and HRD1 polypeptides or fragments thereof as described above. In yet other embodiments, the kit can include ATF6, WFS1 and HRD1 polypeptides or fragments thereof as described above. The kit can further comprise informational material, e.g., instructions for using the kit to identify compounds that modulate protein-protein interactions between, e.g., WFS1 and ATF6 polypeptides, WFS1 and HRD1 polypeptides, ATF6 and HRD1 polypeptides, or WFS1, HRD1 and ATF6 polypeptides, e.g., instructions for how to perform the screening assays described above. The informational material can be descriptive, instructional, marketing or other material that relates to the screening methods described herein and/or the use of WFS1, ATF6, and HRD1 polypeptides for the screening methods described herein.

The informational material of the kit is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is contact information, e.g., a physical address, email address, website, or telephone number, where a user of the kit can obtain substantive information about WFS1, ATF6 and HRD1 and/or their use in the screening methods described herein. Of course, the informational material can also be provided in any combination of formats.

In addition to WFS1, ATF6, and HRD1 polypeptides, the kit can include other ingredients, such as a solvent or buffer, and/or other agents for practicing the screening methods described herein. In such embodiments, the kit can include instructions for using WFS1, ATF6, and HRD1 polypeptides together with the other ingredients.

WFS1, ATF6, and HRD1 polypeptides can be provided in any form, e.g., liquid, dried or lyophilized form. These can be provided in, e.g., substantially pure and/or sterile form. When WFS1, ATF6, and HRD1 polypeptides are provided in a liquid solution, the liquid solution can be an aqueous solution, e.g., a sterile aqueous solution.

The kit can include one or more containers for the composition containing an WFS1 polypeptide, an ATF6 polypeptide, or an HRD1 polypeptide. The kit can include separate containers, dividers or compartments for the composition and informational material. For example, the composition can be contained in a bottle, vial, or syringe, and the informational material can be contained in a plastic sleeve or packet. The separate elements of the kit can be contained within a single, undivided container. For example, the composition can be contained in a bottle, vial or syringe that has attached thereto the informational material in the form of a label. The kit may include a plurality (e.g., a pack) of individual containers, each containing one composition including a WFS1 polypeptide, an ATF6 polypeptide, or an HRD1 polypeptide. For example, the kit can include a plurality of syringes, ampoules, foil packets, or blister packs, each containing a composition including a WFS1 polypeptide, an ATF6 polypeptide, or an HRD1 polypeptide. The containers of the kits can be air tight and/or waterproof.

VI. Pharmaceutical Compositions and Methods of Administration

Compounds useful in treating ER stress disorders, e.g., compounds identified in screens described herein, can be incorporated into pharmaceutical compositions. Such compositions typically include the compound and a pharmaceutically acceptable carrier. A “pharmaceutically acceptable carrier” can include solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be achieved by including an agent which delays absorption, e.g., aluminum monostearate and gelatin in the composition.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

Therapeutic compounds comprising nucleic acids can be administered by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al., Clin. Immunol. Immunopathol. 88(2), 205-10 (1998). Liposomes (e.g., as described in U.S. Pat. No. 6,472,375) and microencapsulation can also be used. Biodegradable targetable microparticle delivery systems can also be used (e.g., as described in U.S. Pat. No. 6,471,996). In some embodiments, targeted delivery of a composition comprising a nucleic acid is used, e.g., to deliver a therapeutic gene to a selected tissue, e.g., the pancreas. For example, local delivery, e.g., by infusion to the selected tissue, can be used. In addition, cells, preferably autologous cells, can be engineered to express a selected gene sequence (e.g., AATF or WFS1, or functional fragments thereof), and can then be introduced into a subject in positions appropriate for the amelioration of the symptoms of an ER stress-related disorder, e.g., exocrine pancreatic cells inserted into the pancreas to treat diabetes. Alternately, cells from a MHC matched individual can be utilized. The expression of the selected gene sequences is typically controlled by appropriate gene regulatory sequences to allow expression in the necessary cell types. Such gene regulatory sequences are well known to the skilled artisan. Such cell-based gene expression techniques are well known to those skilled in the art, see, e.g., Anderson, U.S. Pat. No. 5,399,349.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the patient to be treated; each unit containing a predetermined quantity of active compound calculated to produce the desired therapeutic effect in association with the required pharmaceutical carrier.

The pharmaceutical compositions can be included in a container, pack, or dispenser together with instructions for administration.

Toxicity and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred.

Data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The terms “effective amount” and “effective to treat,” as used herein, refer to an amount or a concentration of a compound utilized for a period of time (including acute or chronic administration and periodic or continuous administration) that is effective within the context of its administration for causing an intended effect or physiological outcome. Effective amounts of compound for use in the present invention include, for example, amounts that, e.g., modulate ER stress signaling, inhibits ER-stress associated cell death, or generally improve the prognosis of a patient diagnosed with an ER stress disorder. The term “treat(ment)” is used herein to describe delaying the onset of, inhibiting, or alleviating the detrimental effects of a condition, e.g., an ER stress disorder.

For the compounds described herein, an effective amount, e.g. of a small molecule, protein or polypeptide (i.e., an effective dosage), ranges from about 0.001 to 30 mg/kg body weight, e.g. about 0.01 to 25 mg/kg body weight, e.g. about 0.1 to 20 mg/kg body weight. The compound can be administered, e.g., one time per week for between about 1 to 10 weeks, e.g. between 2 to 8 weeks, about 3 to 7 weeks, or for about 4, 5, or 6 weeks. In certain cases, the compound can be administered for a period of years, e.g., one to three times per week for between 1 to 30 years, e.g., between 2 to 20 years, about 5 to 15 years, or for about 10, 15, or 30 years. The skilled artisan will appreciate that certain factors influence the dosage and timing required to effectively treat a patient, including but not limited to the type of patient to be treated, the severity of the disease or disorder, previous treatments, the general health and/or age of the patient, and other disorders present. Moreover, treatment of a patient with a therapeutically effective amount of a protein, polypeptide, antibody, or other compound can include a single treatment, or can include a series of treatments.

### EXAMPLES

### Example 1

### AATF Protects Cells from ER-Stress Mediated Apoptosis

This example demonstrates that AATF protects cells from ER stress-mediated apoptosis through transcriptional regulation of Akt1. Accordingly, AATF is a potential new target for the treatment of ER stress disorders such as diabetes and neurodegenerative disorders.

Materials and Methods

Cell culture and transfection of small interfering RNA. Rat insulinoma cells, INS-1 832/13, were a gift from Dr. Christopher Newgard (Duke University Medical Center). These cells were cultured in RPMI 1640 supplemented with 10% FBS. Mouse embryonic fibroblasts, COS7 cells, and Neuro2a cells were maintained in DMEM with 10% fetal bovine serum. Human neuroblastoma cells, SH-SY5Y cells, were cultured in DMEM/F12 with 10% fetal bovine serum.

The Cell Line Nucleofector™ Kit V with a Nucleofector Device (Amaxa Biosystems, Gaithersburg, Md.) was used to transfect small interfering RNA (siRNA) for WFS1, AATF, and Akt1 into INS1 and SH-SY5Y cells. At QIAGEN (Valencia, Calif.), siRNAs for rat and human AATF, and rat Akt1 were designed and synthesized:

| | | |
|---|---|---|
| rat AATF: | AAGCGCTCTGCCTACCGAGTT | (SEQ ID NO: 1) |
| human AATF: | AAGCGCTTTGCCGACTTTACA | (SEQ ID NO: 2) |
| rat Aktl: | AACGCCTGAGGAGCGGGAAGA | (SEQ ID NO: 3) |

Cell viability and cell death assay. SH-SY5Y cells transduced with lentivirus expressing α-synuclein or GFP were cultured in 24-well plates for 16 hours, and then 3-(4,5-dimethylthiazol-2-yl)-5-(3-carboxymethoxyphenyl)-2-(4-sulfophenyl)-2H-tetrazolium inner salt (MTS) assays using Cell Titer 96 (Promega) were performed.

Cell toxicity was analysed at the same time by using the ToxiLight kit (Lonza, Allendale, N.J.) according to the manufacturer's protocol to measure the release of adenylate kinase from damaged cells into the culture medium.

GeneChip Array Analysis. Rat insulinoma cells, INS-1 832/13, were transfected with siRNAs against Aatf. Cells with siRNA against Aatf were treated with 0.5 μM of thapsigargin for 16 hr after transfection. Total RNA was isolated for each sample and processed for GeneChip analysis by the Whitehead Institute Center for microarray technology (Cambridge, Mass.). The final product was hybridized to the GeneChip® Rat Genome 230 2.0 Arrays (Affymetrix, Santa Clara, Calif.) and scanned with a GeneChip Scanner 3000.

Array analysis was done using BRB-ArrayTools Version 3.6.0 Beta, developed by Dr. Richard Simon and Amy Peng Lam. The robust multichip analysis algorithm (RMA) was used for reduction of probe intensities into probe set values. Samples treated with siRNA against AATF (n=3) were compared to control samples (n=3) using a random-variance t-test. This test permits the sharing of information among genes about within-class variation without assuming that all genes have the same variation (see, e.g., Wright, G. W. & Simon, R. M., Bioinformatics 19, 2448-2455 (2003)). A gene was considered to be statistically significant if the p-value was less than 0.002.

Immunoblotting. Cells were lysed for 15 min on ice at 4° C. in ice-cold M-PER buffer (PIERCE, Rockford, Ill.) containing protease inhibitors. The lysates were then cleared by centrifuging the cells at 13,000 g. Lysates were normalized for total protein, separated by SDS-PAGE (15% gel or 5%-20% gradient gel), and transferred onto a polyvinylidene difluoride membrane. To detect human AATF protein, anti-AATF antibody from Bethyl (Montgomery, Tex.) was used. To detect rat AATF protein, a rabbit anti-AATF antibody generated using a peptide, RPREADPEADPEEATR, was used. Anti-actin and anti-myc (9E10) antibodies were purchased from Sigma (St. Louis, Mo.); anti-eIF2α was purchased from Santa Cruz Biotechnology (Santa Cruz, Calif.); anti-phospho-eIF2α, anti-Akt, anti-Akt1, anti-phospho-Akt, anti-Creb, anti-tubulin, and anti-caspase-3 antibodies were purchased from Cell Signaling (Danvers, Mass.).

Lentivirus system. Mouse AATF, mouse Akt1, and human α-synuclein cDNAs were purchased from Open Biosystems (Huntsville, Ala.). Their cds portions were subeloned into lentiviral expression vectors. For mouse AATF and mouse Akt1, pLenti-CMV/TO and for human α-synuclein, pLenti-CMV/TO, these were kind gifts from Dr. Eric Campeau at the University of Massachusetts Medical School. Lentiviral particles were produced in HEK293T cells by transfection using Lipofectamine-2000 (Invitrogen, Carlsbad, Calif.). Lentiviral-containing supernatant was collected 48 hr after transfection and stored at −80° C. To establish a cell line that constitutively expressed the tetracycline repressor, INS-1 832/13 cells were infected with pLenti-TetR, followed by blasticidine selection (a kind gift from Dr. Eric Campeau). These cells were then infected overnight with inducible lentiviruses (pLenti-CMV/TO-AATF or pLenti-CMV/TO-Akt1). After letting cells recover in fresh medium for 24 hr, puromycine was added (2 μg/mL) to select for transfected cells. To induce AATF in INS-1 832/13 cells, 2 μg/ml of doxycycline was added to the medium, which was then incubated for 48 hr. For Akt1 expression, 4 ng/ml of doxycycline was added to the medium, which again was incubated for 48 h. This amount was determined to express 1-2 fold of endogenous Akt1 in INS-1 832/13 cells. To establish cells that constitutively express α-synuclein, SH-SY5Y cells were infected with lentivirus (pLenti-CMV-α-synuclein), which was followed by G418 selection.

Real-time polymerase chain reaction. Total RNA was isolated from cells using RNeasy Mini Kit (Qiagen) and reverse transcribed using 1 μg of total RNA from cells with Oligo-dT primer. For the thermal cycle reaction, the iQ5 system (BioRad, Hercules, Calif.) was used at 95° C. for 10 min, 40 cycles at 95° C. for 10 sec, and at 55° C. for 30 sec.

The relative amount for each transcript was calculated by a standard curve of cycle thresholds for serial dilutions of cDNA samples and normalized to the amount of actin. The polymerase chain reaction (PCR) was done in triplicate for each sample, after which all experiments were repeated twice. The following sets of primers and Power SYBR Green PCR Master Mix (Applied Biosystems, Foster City, Calif.) were used for real-time PCR:

| | | |
|---|---|---|
| human | GAAAGATGCTGCGTTCCGA | GGGTTGGGCGGGACA |
| AATF | (SEQ ID NO: 4) | (SEQ ID NO: 5) |
| human | ACCATGGATGATGATATCGCC | GCCTTGCACATGCCGG |
| actin | (SEQ ID NO: 6) | (SEQ ID NO: 7) |
| mouse | TTCTTGGCAAACCGGAGC | AGCGTCTCTGGTTCTCCTGG |
| AATF | (SEQ ID NO: 8) | (SEQ ID NO: 9) |
| mouse | GCAAGTGCTTCTAGGCGGAC | AAGAAAGGGTGTAAAACGCA |
| actin | (SEQ ID NO: 10) | GC |
| | | (SEQ ID NO: 11) |
| mouse | TTCAGCCAATTATCAGCAAACTCT | TTTTCTGATGTATCCTCTTCAC |
| BiP | (SEQ ID NO: 12) | CAGT |
| | | (SEQ ID NO: 13) |
| mouse | CCATCAACATGCTCCCGTTC | GGGTAGGCCTCGCCATACA |
| WFS1 | (SEQ ID NO: 14) | (SEQ ID NO: 15) |
| mouse | GCTCTCTCCAAAGTGCTTCCA | TGCATCCTGAACTTTATTCCCA |
| Erolα | (SEQ ID NO: 16) | (SEQ ID NO: 17) |
| mouse | CCACCACACCTGAAAGCAGAA | AGGTGAAAGGCAGGGACTCA |
| Chop | (SEQ ID NO: 18) | (SEQ ID NO: 19) |
| mouse | TGGCCGGGTCTGCTGAGTCCG | GTCCATGGGAAGATGTTCTGG |
| total | (SEQ ID NO: 20) | (SEQ ID NO: 21) |
| XBP-1 | | |
| mouse- | CTGAGTCCGAATCAGGTGCAG * | GTCCATGGGAAGATGTTCTGG |
| spliced | (SEQ ID NO: 22) | (SEQ ID NO: 23) |
| XBP-1 | | |
| rat AATF | CCGAGTTCTTGGCAAACCTG | TCTCCGGTTCTCCTGGCA |
| | (SEQ ID NO: 24) | (SEQ ID NO: 25) |
| rat actin | GCAAATGCTTCTAGGCGGAC | AAGAAAGGGTGTAAAACGCA |
| | (SEQ ID NO: 26) | GC |
| | | (SEQ ID NO: 27) |
| rat BiP | TGGGTACATTTGATCTGACTGGA | CTCAAAGGTGACTTCAATCTG |
| | (SEQ ID NO: 28) | GG |
| | | (SEQ ID NO: 29) |
| rat Chop | AGAGTGGTCAGTGCGCAGC | CTCATTCTCCTGCTCCTTCTCC |
| | (SEQ ID NO: 30) | (SEQ ID NO: 31) |
| rat total | TGGCCGGGTCTGCTGAGTCCG | ATCCATGGGAAGATGTTCTGG |
| XBP-1 | (SEQ ID NO: 32) | (SEQ ID NO: 33) |
| rat- | CTGAGTCCGAATCAGGTGCAG * | ATCCATGGGAAGATGTTCTGG |
| spliced | (SEQ ID NO: 34) | (SEQ ID NO: 35) |
| XBP-1 | | |
| rat WFS1 | ATCGACAACAGCGCCGA | GCATCCAGTCACCCAGGAAG. |
| | (SEQ ID NO: 36) | (SEQ ID NO: 37) |

* The original CAG sequence was mutated to AAT to reduce the background signal from unspliced XBP-1.

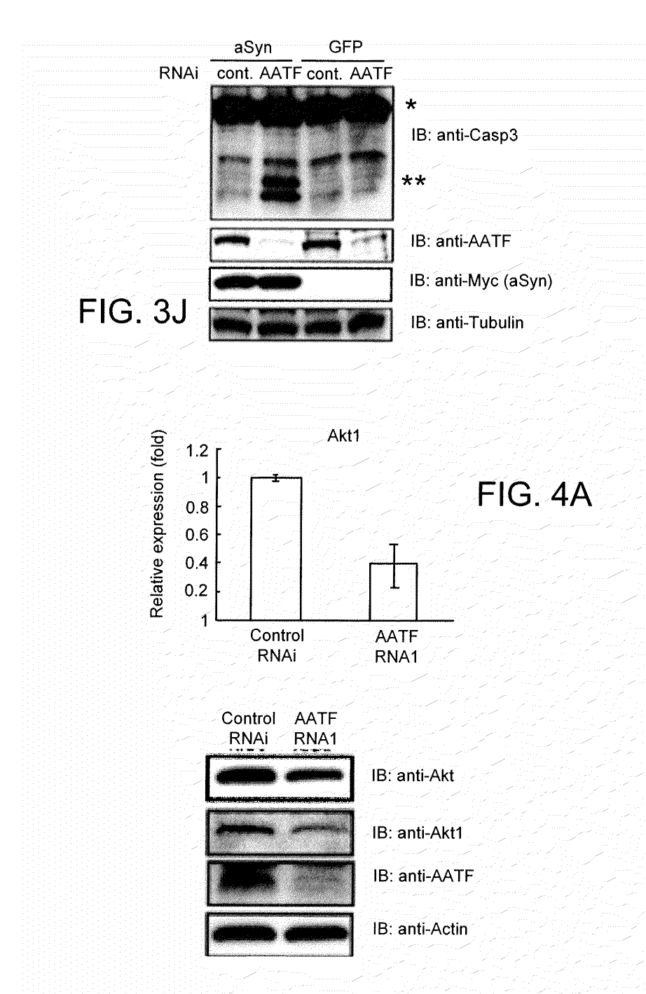

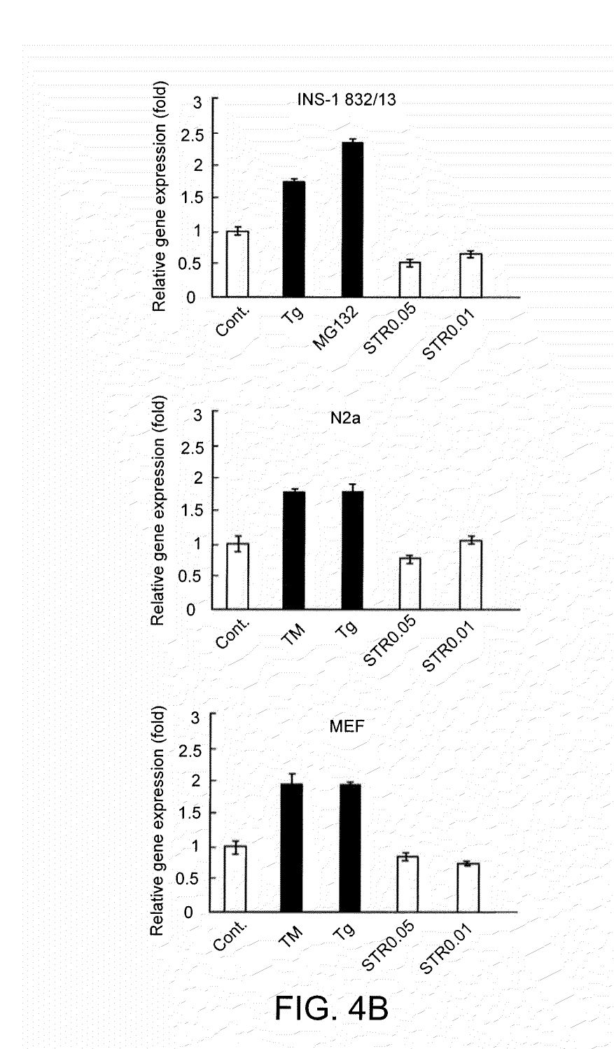

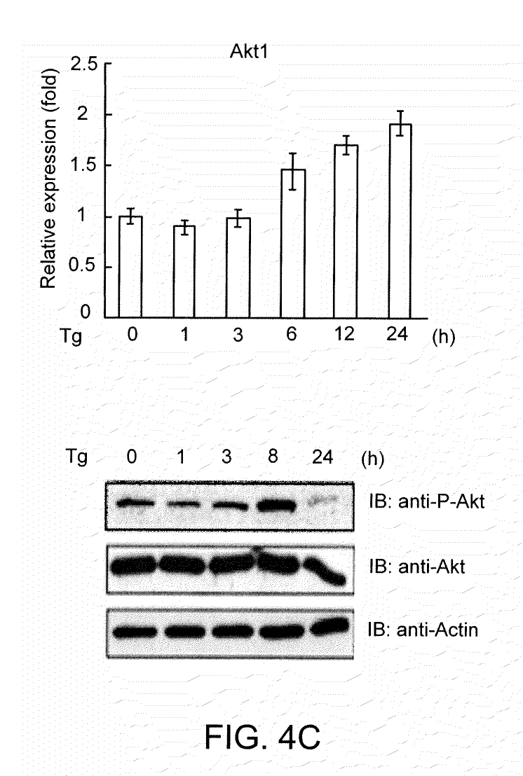

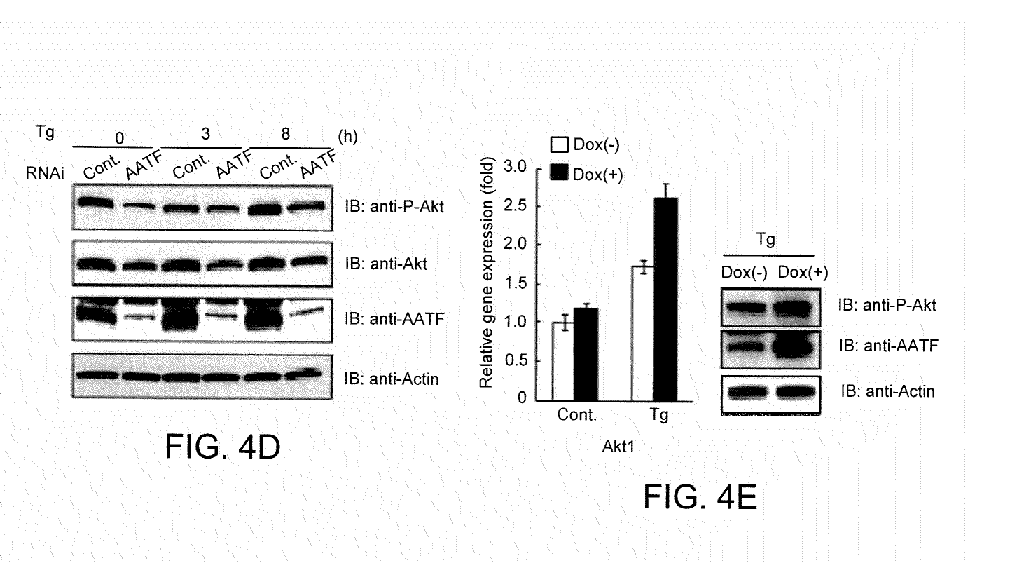

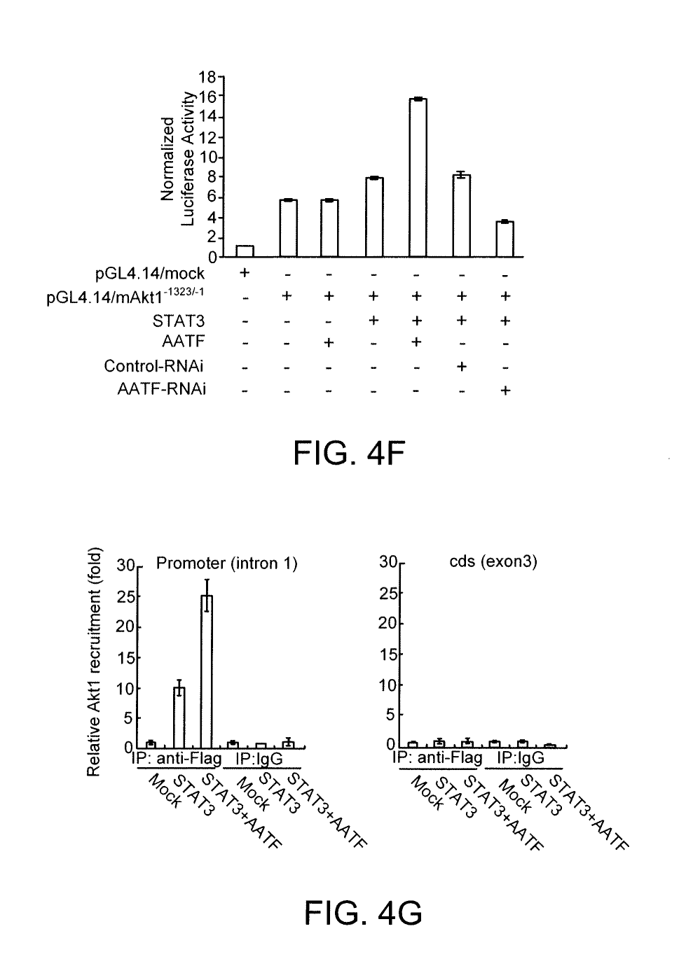

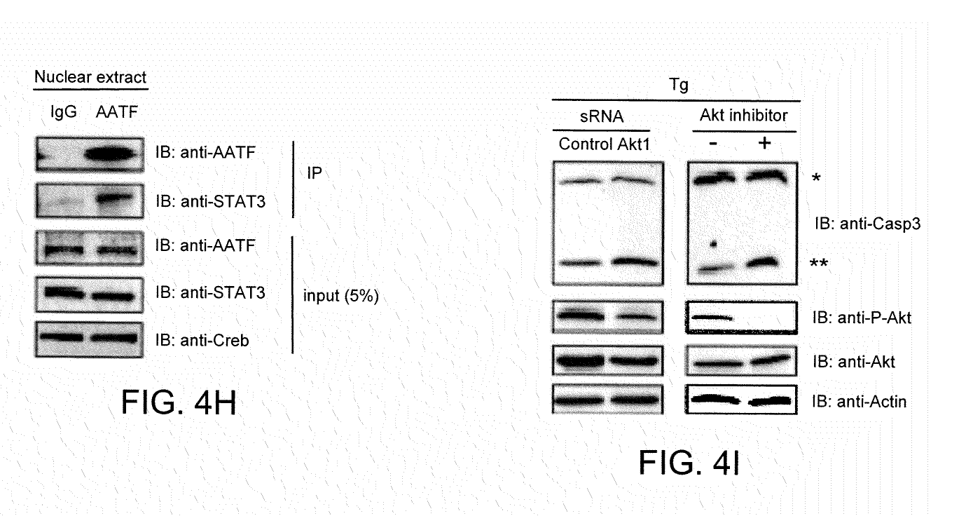

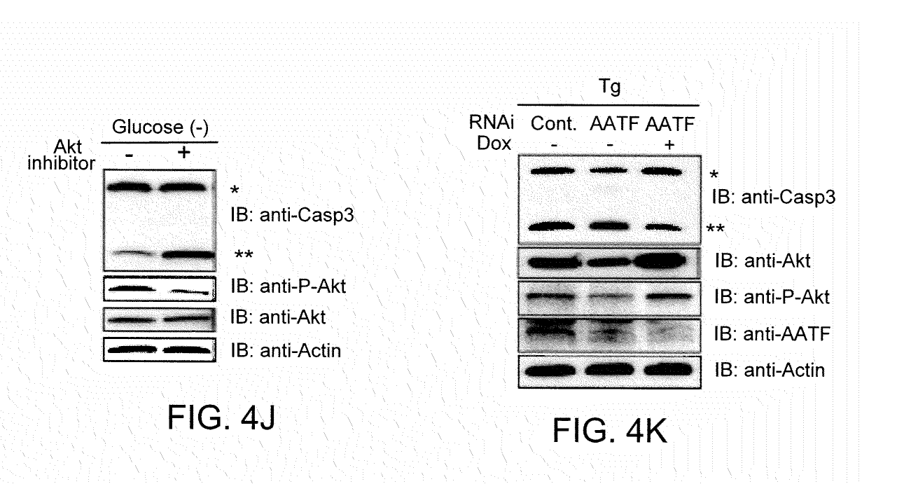

Statistical analysis. Two-way ANOVA was done to determine the main effect of AATF RNAi, the main effect of TQ and the interaction between AATF RNAi and thapsgargin (FIG. 4B). When there was a significant interaction (p<0.05), a set of predetermined contrasts was performed in the framework of one-way ANOVA. Two-way ANOVA was done to determine the main effect of doxycycline, the main effect of thapsigargin, and the interaction between doxycycline and thapsigargin (FIG. 4E). When there was a significant interaction (p<0.05), a set of predetermined contrasts was performed. As shown in FIGS. 4B and 4E, cell death (y) was measured as a proportion of dead cells among all cells treated. Arcsine(sqrt(y)) transformation was frequently applied to the raw data to homogenize the variance before further data analysis (see, e.g., Freeman, M. F. & Tukey, J. W., Ann Mathem Stat 21, 607-611 (1950)). However, results in this dataset were similar with or without transformation. Therefore, for ease of interpretation, only results using untransformed data are presented. Two-way ANOVA was used to determine the main effect of AATF RNAi, the main effect of α-synuclein, and the interaction between AATF RNAi and α-synuclein. When there was a significant interaction (p<0.05), a set of predetermined contrasts was done.

TUNEL assay. Apoptotic cell death was assessed by the TUNEL assay. Apoptotic cells were counted using the DeadEnd™ Colorimetric TUNEL System (Promega, Madison Wis.). Counting was done by an investigator who was blind to the experimental condition.

MTS assay and Cell toxicity assay. SH-SY5Y cells transduced with lenti-virus expressing α-synuclein or GFP were cultured in 24-well plates for 16 hr. 3-(4,5-dimethylthiazol-2-yl)-5-(3-carboxymethoxyphenyl)-2-(4-sulfophenyl)-2H-tetrazolium inner salt (MTS) assays were then performed using Cell Titer 96 (Promega).

Cell toxicity was analyzed at the same time by measuring the release of adenylate kinase from damaged cells into the culture medium by using the ToxiLight kit (Lonza, Allendale, N.J.) according to the manufacturer's protocol.

Promoter assay. Several fragments of the promoter region of mouse Akt1 were amplified by PCR and cloned into the KpnI/XhoI site of the pGL4.14 vector (Promega). Since among these fragments of pGL4.14/Akt1^{-1323/-32} had the best relative activity when co-expressed with constitutive STAT3 (data not shown), it was used for further promoter assay. Akt1^{-1323/-32} contains 5 putative STAT3 sites that correspond to TT(N)₄₋₆AA: −1245/−1237, −1034/−1026, −653/−644, −420/−413, and −392/−385. To construct pCS2+MT/AATF, the coding regions of mouse AATF were amplified and cloned into EcoRV/XhoI site of pCS2+MT vector. pFlag-STAT3-C vector which expressed constitutive form of STAT3 was obtained from addgene (Addgene Inc., Cambridge, Mass.). N2a cells were transfected with pGL4.14/Akt1^{-13231/-32}, pFlag/STAT3-C(STAT3), pCS2+/AATF (AATF), siRNA directed against AATF, and β-garactosidase. pGL4.14/mock and control scrambled siRNA were used as negative controls. After 24-hr incubation, luciferase activities were measured using the Luciferase assay system (Promega). β-garactosidase activity was measured by b-Gal Reporter Gene Assay, chemiluminescent (Roche Diagnostics, Mannheim, Germany). The assay was performed independently three times.

Chromatin immunoprecipitaion (ChIP). HEK293T cells were transfected with pFlag-STAT3-C with or without pCS2+/AATF. Cells were fixed after 24-hr of incubation. ChIPs were performed as described before. Purified DNA from crosslinked cells was dissolved in 50 μl TE; 3 μl was used for PCR. Inputs consisted of 1% of chromatin before immunoprecipitation. Quantitative PCRs were performed as described in Real-time polymerase chain reaction section using the following primer sets:

mouse Akt1 promoter (intron1):

(SEQ ID NO: 38)

TCCCTCTGGAAGAGAAGCAA
and

(SEQ ID NO: 39)

TAGCTAGCCTGTGCAAAGCA;
mouse Akt1 cds (exon3):

(SEQ ID NO: 40)

ATGGACTCAAGAGGCAGGAA
and

(SEQ ID NO: 41)

TCTTCAGCCCCTGAGTTGTC.

Results

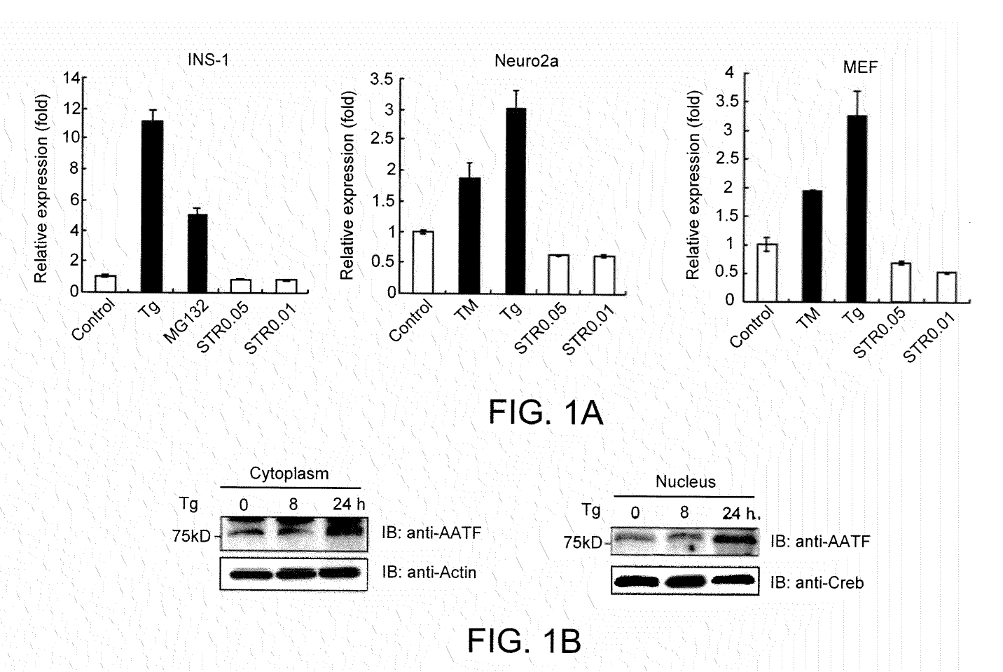

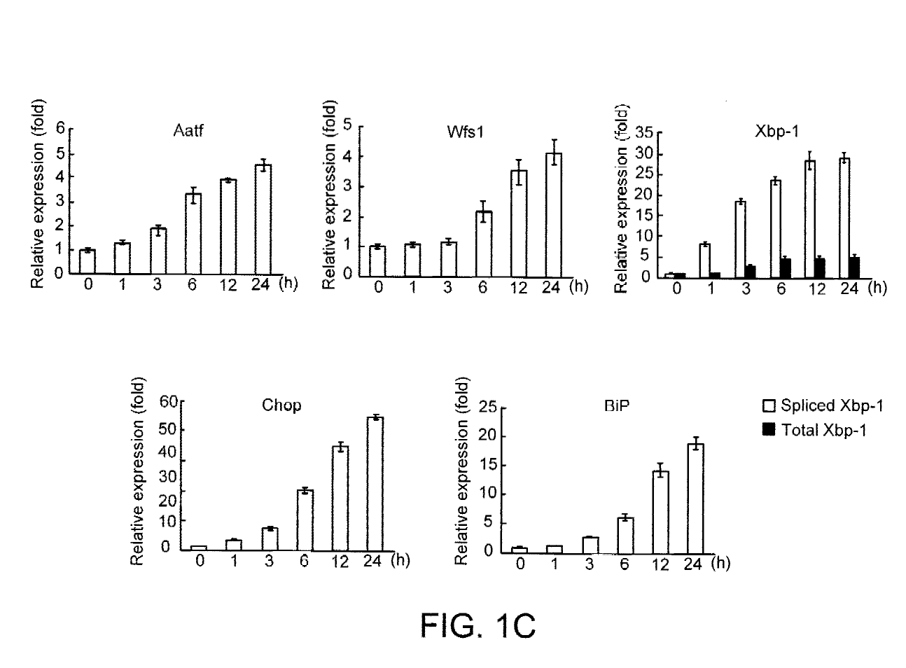

To investigate a possible role for AATF in the ER stress signaling, also called the Unfolded Protein Response (UPR), the expression levels of AATF mRNA in INS-1 832/13 cells, neuro2A cells, and mouse embryonic fibroblasts treated with various ER stress inducers were measured. As shown in FIG. 1A, AATF mRNA was up-regulated by ER stress inducers, including tunicamycin, thapsigargin, and MG132, but not by a general apoptosis inducer, staurosporin, indicating that AATF expression is specifically increased by ER stress. The up-regulation of AATF by ER stress was confirmed using both cytoplasmic and nuclear protein extracts from INS-1 832/13 cells (FIG. 1B). After treating these cells with thapsigargin, the up-regulation of AATF mRNA by ER stress was compared to other ER stress markers, including BiP, Chop, XBP-1, and WFS1. As shown in FIG. 1C, AATF mRNA expression continued to increase up to 24 hr after the initiation of ER stress.

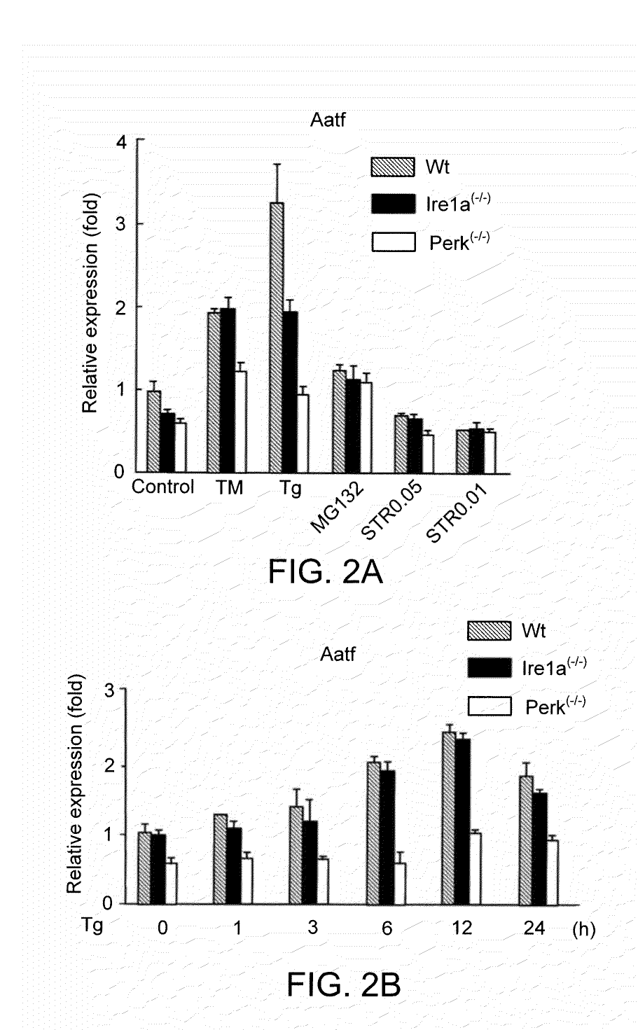

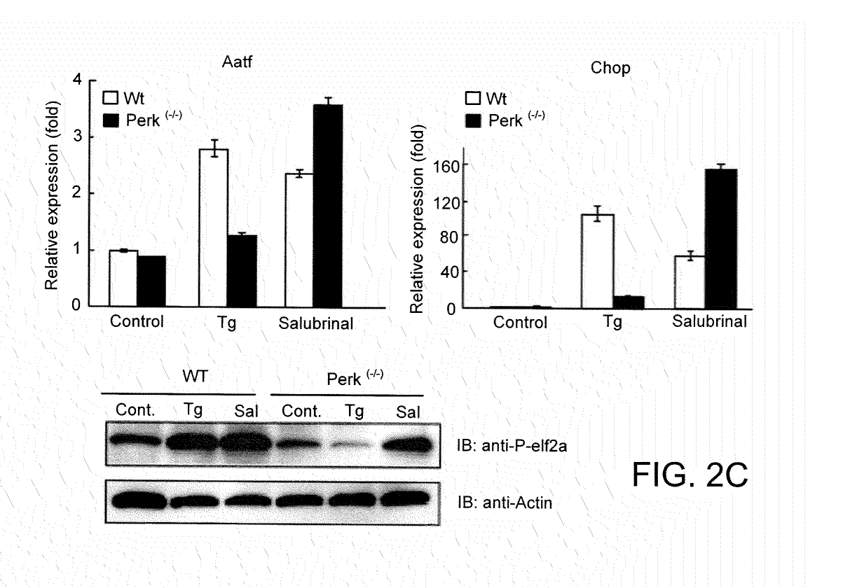

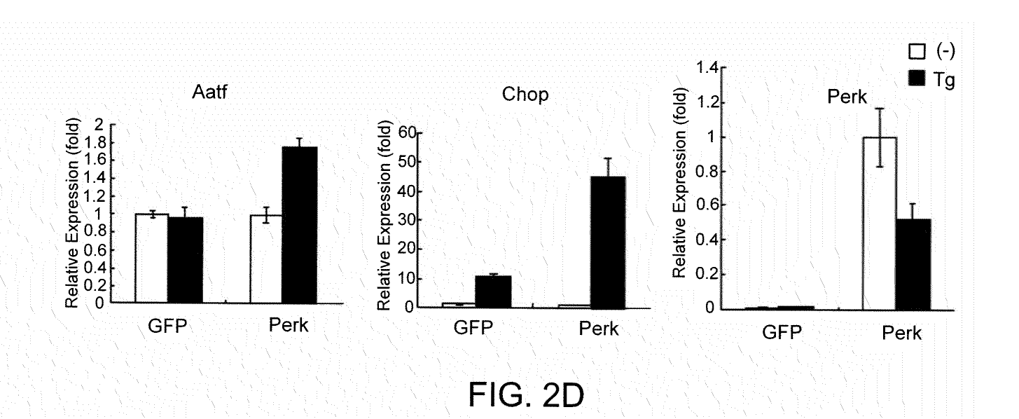

The pathway by which AATF expression is regulated was investigated. IRE 1 and PERK are ER-resident protein kinases and regulators of the UPR. The expression levels of AATF were measured by real-time PCR in Ire1α^{-/-} and Perk^{-/-} mouse embryonic fibroblasts under ER stress conditions. In wild-type fibroblasts, expression levels of AATF mRNA were increased 2-3 fold by tunicamycin and thapsigargin, whereas the induction of AATF was attenuated in Perk^{-/-} mouse embryonic fibroblasts, but not in Ire1α^{-/-} cells, suggesting that PERK regulates AATF expression (FIGS. 2A, B). To confirm this, Perk^{-/-} mouse embryonic fibroblasts were transfected with PERK expression plasmid, then AATF gene expression was measured. Chop expression was also measured as a control. PERK expression could restore both AATF and Chop expression (FIG. 2D). These results indicate that PERK signaling regulates AATF expression at the transcription level.

It is established that PERK-mediated eIF2α phosphorylation has role in the up-regulation of its target genes (see, e.g., Harding, H. P., et al., Mol Cell 6, 1099-1108 (2000); Harding et al., Nature 397, 271-274 (1999)). Whether eIF2α phosphorylation could increase AATF expression was determined. Wild-type and Perk^{-/-} mouse fibroblasts were treated with salubrinal, a compound that increases eIF2α phosphorylation (see Boyce, M., et al., Science (New York, N.Y. 307, 935-939 (2005)), then AATF expression was measured, with Chop expression as a control. AATF expression, as well as Chop expression, was increased by salubrinal treatment in both wild-type and Perk^{-/-} mouse embryonic fibroblasts (FIG. 2C, upper panel). Immunoblot confirmed that eIF2α phosphorylation levels were increased by salubrinal (FIG. 2C, lower panel).

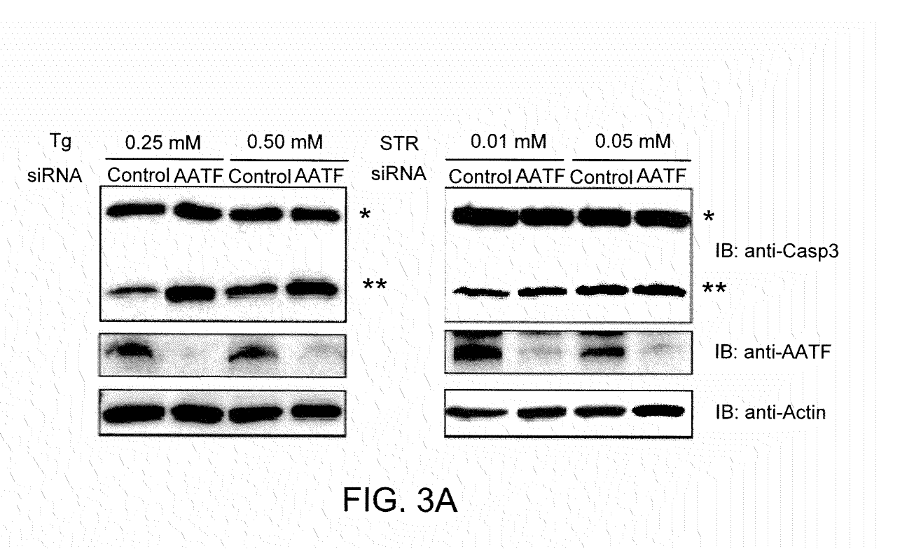

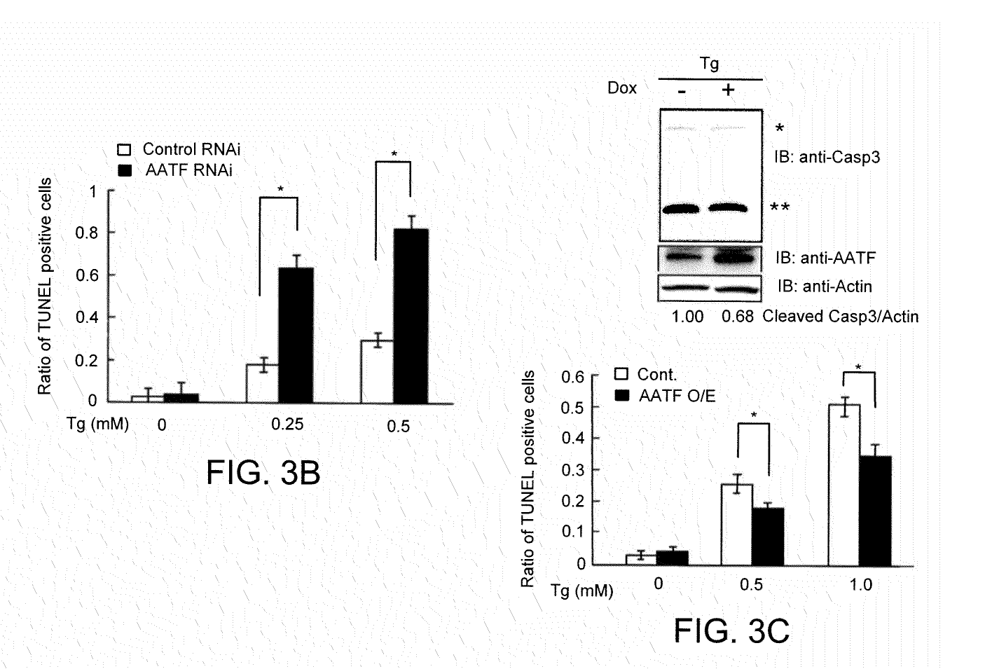

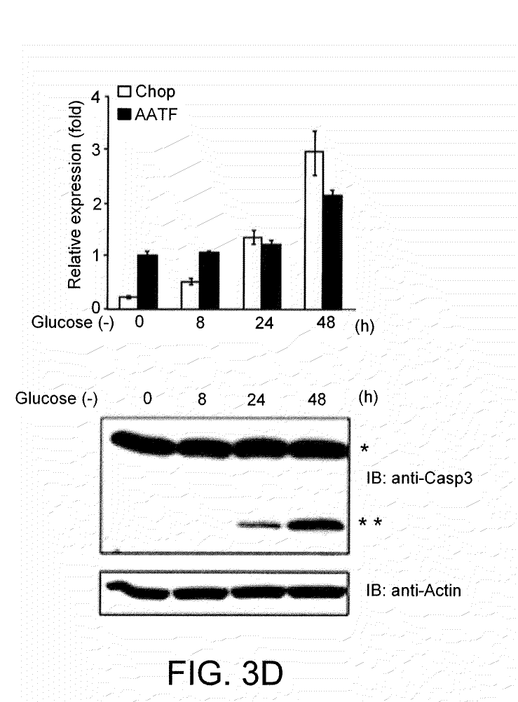

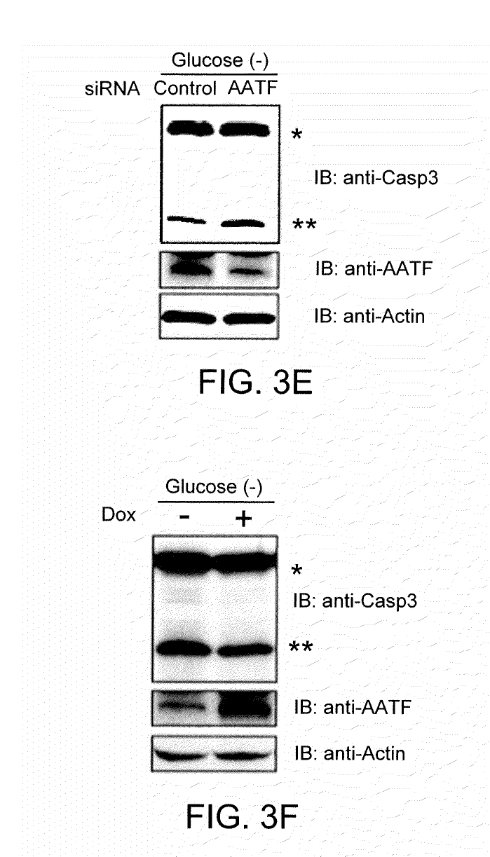

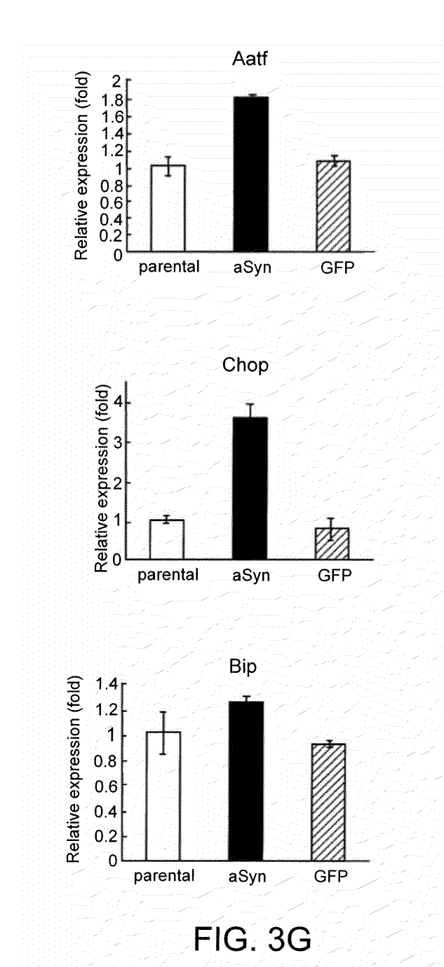

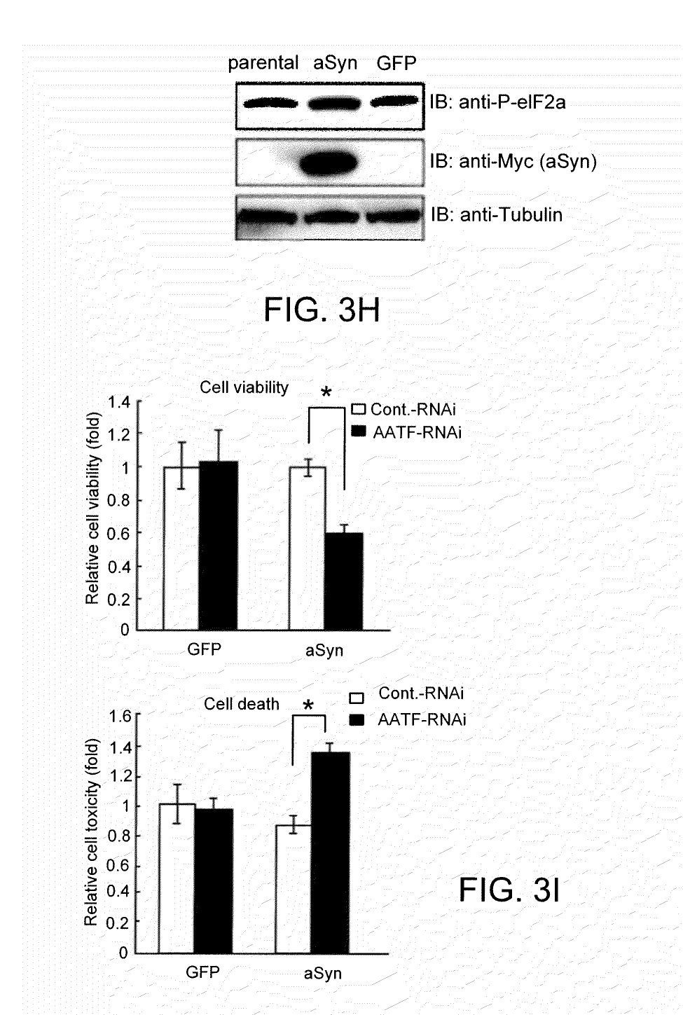

The ability of AATF to protect cells from ER stress-mediated apoptosis was examined. INS-1 832/13 cells were transfected with siRNA directed against AATF, then challenged with thapsigargin or staurosporin, and the cleavage of caspase-3, a marker for apoptosis, was measured. As compared to control cells, cells transfected with siRNA directed against AATF showed increased cleavage of caspase-3 by thapsigargin, but not staurosporin, demonstrating that inactivation of AATF rendered cells specifically sensitive to ER stress-mediated apoptosis (FIG. 3A). To confirm this, apoptosis in AATF-knockdown cells using TUNEL staining was measured. FIG. 3B shows that AATF suppression increased the number of TUNEL-positive cells in the presence of ER stress. Whether AATF over-expression would render INS-1 832/13 cells resistant to ER stress-mediated apoptosis was examined. AATF induction using a doxycyline-induced expression system decreased caspase-3 cleavage by thapsigargin (FIG. 3C, upper panel). To confirm this, apoptosis in these cells using TUNEL staining was measured. FIG. 3C (lower panel) shows that AATF induction decreased the number of TUNEL-positive cells.

The role of AATF in protecting cells from ER stress-mediated apoptosis was further examined using a more physiological ER stress inducer, glucose deprivation (see Kozutsumi et al., Nature 332, 462-464 (1988)). Whether glucose deprivation causes ER stress and AATF up-regulation was investigated. INS-1 832/13 cells were cultured in glucose-free medium, then expression levels of Chop and AATF, as well as capase-3 cleavage, were measured. It was shown that glucose starvation increased Chop and AATF expression, as well as caspase-3 cleavage, indicating that glucose starvation induces ER stress-mediated apoptosis (FIG. 3D). AATF-knockdown INS-1 832/13 cells were challenged with glucose starvation. AATF-knockdown sensitized INS-1 832/13 cells to glucose deprivation-mediated apoptosis (FIG. 3E). In addition, AATF over-expression using doxycycline-mediated induction decreased caspase-3 cleavage caused by glucose deprivation in INS-1 832/13 cells (FIG. 3F). These results indicate that AATF functions in protecting cells from ER stress-mediated apoptosis.

Accumulation of α-synuclein in Lewy bodies and neurites is a pathological hallmark in Parkinson's disease (see, e.g., Lee et al., Neuron 52, 33-38 (2006)). It has been established that over-expression of α-synuclein elicits ER stress and subsequently causes ER stress-mediated neuronal cell death, suggesting that the balance between anti-apoptotic and pro-apoptotic components of the ER stress signaling network is a determinant of α-synuclein-mediated neuronal cell death (see Smith, W. W., et al., Hum Mol Genet. 14, 3801-3811 (2005); Cooper, A. A., et al., Science (New York, N.Y. 313, 324-328 (2006)). To determine whether AATF is involved in csynuclein-mediated neuronal cell death, expression levels of AATF mRNA in SH-SY5Y cells overexpressing α-synuclein were determined. As compared to expression in control cells, AATF, as well as Chop, a major pro-apoptotic component of the UPR, was increased in cells expressing α-synuclein, indicating that both anti-apoptotic and pro-apoptotic components of the UPR are activated in SH-SY5Y cells expressing α-synuclein (FIG. 3G). Immunoblot analysis showed that eIF2α phosphorylation was also increased in the cells expressing α-synuclein (FIG. 3H).

Whether the reduction of AATF expression makes SH-SY5Y cells sensitive to α-synuclein-mediated cell death was investigated. SH-SY5Y cells expressing α-synuclein were transfected with siRNA directed against AATF, cell viability and death were determined. It was shown that suppression of AATF expression decreased viability (FIG. 3I, left panel) and increased apoptosis (FIG. 3I, right panel) in the cells expressing α-synuclein as compared to control cells. To confirm this, the cleavage of caspase-3 was also measured. FIG. 3J) shows that AATF-knockdown increased the cleavage of caspase-3 in the cells expressing α-synuclein, but not in control cells.

AATF has an L-zip domain in the N-terminal, followed by two nuclear localization signals in the C-terminal and has been proposed to play an important role in transcription. Immunostaining in COS7, INS1 832/13, and primary neurons revealed that AATF was enriched in the nucleus and nucleolus in various cell types (data not shown) as reported (see Thomas et al., Dev Biol 227, 324-342 (2000); Guo, Q. & Xie, J., The Journal of Biological Chemistry 279, 4596-4603 (2004)).

To identify transcriptional targets of AATF, gene expression profiles were examined using DNA microarray in AATF-knockdown INS-1 832/13 cells and control INS-1 832/13 cells transfected with scramble siRNA and treated with thapsigargin. Genes that were significantly down regulated (p<0.002) more than two-fold by AATF siRNA were defined as AATF targets under ER stress conditions (Table 2). Eight target genes were identified, including a survival kinase, Akt1, which protects cells from apoptosis under various conditions (see, e.g., Amaravadi, R. & Thompson, C. B., The Journal of Clinical Investigation 115, 2618-2624 (2005)).

TABLE 2

| | | AATF siRNA/ | | |
|---|---|---|---|---|
| Affymetrix | | Control Fold | Gene | |
| ID | p-value | Change | Symbol | Description |
| 1370910_at | 8.00E−07 | −2.88 | Rfc2 | Replication factor C (activator 1) 2 |
| 1394833_at | 4.69E−05 | −2.61 | — | Transcribed locus (chr4:82679035-82679475[+]) |
| 1376800_at | 5.00E−07 | −2.48 | — | Transcribed locus (chr4:82679035-82679475[+]) |
| 1379374_at | 7.00E−06 | −2.59 | Prg1 | Plasticity related gene 1 |
| 1385981_at | 4.00E−07 | −2.27 | Prg1 | Plasticity related gene 1 |
| 1370166_at | 1.18E−04 | −2.33 | Sdc2 | Syndecan 2 |
| 1382189_at | 2.90E−06 | −2.11 | Sdc2 | Syndecan 2 |
| 1388309_at | 3.00E−07 | −2.30 | Hmga1 | High mobility group AT-hook 1 |
| 1383126_at | 1.20E−05 | −2.19 | Akt1 | Thymoma viral proto-oncogene 1 |
| 1368862_at | 1.71E−03 | −2.10 | Akt1 | Thymoma viral proto-oncogene 1 |
| 1377760_at | 6.00E−07 | −2.11 | Noc41 | Nucleolar complex associated 4 homolog |
| 1374521_at | 2.20E−06 | −2.03 | — | Transcribed locus (chr14:1780721-1781454[−]) |

As shown in FIG. 4A, AATF-knockdown by siRNA suppressed Akt1 mRNA and protein expression. Whether Akt1 expression is increased by ER stress was examined. The expression levels of Akt1 mRNA were measured in the presence of ER stress in INS-1 832/13, neuro2A, and mouse embryonic fibroblasts. FIG. 4B shows that Akt1 mRNA expression was increased 1.5-2 fold by various ER stress inducers, including tunicamycin, thapsigargin, and MG132, but not staurosporin. Measuring Akt1 mRNA expression at different times under ER stress conditions, it was found that Akt1 expression was increased during ER stress, with a peak at 24 hr (FIG. 4C, left panel). Collectively, these results indicate that Akt1 is a target for AATF in the presence of ER stress.

It has been proposed that phosphorylation of Akt is important in protecting cells from apoptosis (Srinivasan, S., et al., Diabetes 54, 968-975 (2005). As shown in FIG. 4C (right panel), phosphorylation level of Akt was increased up to 8 hr after treatment, but decreased at 24 hr. To study the relationship between AATF suppression and Akt phosphorylation, AATF expression was suppressed using siRNA directed against AATF in INS-1 832/13 cells and the cells were treated with thapsigargin for 0, 3, and 8 hr, then Akt expression and Akt phosphorylation levels were measured by immunoblot. Both Akt expression and Akt phosphorylation levels were decreased by AATF siRNA (FIG. 4D). To further confirm the relationship between AATF and Akt1 expression, an inducible lentivirus system expressing the AATF gene was generated. INS-1 832/13 cells were infected with the virus and Akt1 expression levels were measured. As shown in FIG. 4E, AATF over-expression enhanced Akt1 mRNA expression under ER stress conditions, leading to an increase in Akt phosphorylation.

Stat3 has been proposed to play an important role in Akt1 expression (see, e.g., Park, S., et al., The Journal of Biological Chemistry 280, 38932-38941 (2005); Xu, Q., et al., Oncogene 24, 5552-5560 (2005)). The role of Stat3 in AATF-mediated induction of Akt1 was investigated. A plasmid expressing Stat3 with or without AATF was co-transfected into 293T cells along with a reporter plasmid containing 1.3 kilobases of the Akt1 promoter driving the luciferase gene. As shown in FIG. 4F, Stat3 expression caused an 8-fold induction of luciferase activity, and siRNA-mediated knockdown of AATF abrogated this induction. The addition of AATF to Stat3 led to a 16-fold induction of luciferase activity (FIG. 4F). Chromatin immunoprecipitation (ChIP) analysis verified that Stat3 bound to the Akt1 promoter in response to AATF expression (FIG. 4G). Further, as shown in FIG. 4H, Stat3 and Akt1 interacted in the nucleus.

To study the involvement of the Akt1 pathway in protecting cells from ER stress-mediated apoptosis, the pathway was suppressed in INS1 832/13 cells using siRNA directed against Akt1 (FIG. 4I, left panel) or an Akt inhibitor, SH-5, (FIG. 4I, right panel). These cells were then challenged with thapsigargin and the cleavage of caspase-3 was measured. Both Akt1 siRNA and the Akt inhibitor increased cleavage of caspase-3, indicating that Akt1 gene expression and its phosphorylation are active in protecting cells from ER stress-mediated apoptosis (FIG. 4I). To study the involvement of the Akt1 pathway in protecting cells from apoptosis mediated by glucose deprivation, INS1 . 832/13 cells were treated with an Akt inhibitor, SH-5, then challenged with glucose deprivation, and the cleavage of caspase-3 was measured. Akt1 inhibitor treatment increased the cleavage of caspase-3 (FIG. 4J). To determine whether Akt1 over-expression can rescue cells from apoptosis caused by the suppression of AATF, INS-1 832/13 cells were transfected with control siRNA or siRNA against AATF. These cells were then challenged with thapsigargin with or without the induction of Akt1, using the lentivirus-based doxycycline-mediated Akt1 induction system, and caspase-3 cleavage was measured (FIG. 4K). Taken together, these results demonstrate that Akt1 protects cells from ER stress-mediated apoptosis.

### Example 2

### WFS1-Mediated ATF6 Proteolysis Regulates ER Stress Signaling Network

In this example, evidence demonstrates that WFS1 regulates ATF6 transcriptional activity through the proteasome-mediated degradation of ATF6 protein, and that HRD1 is an E3 ligase for ATF6. ATF6 is a mediator of transcriptional induction of the ER stress response genes. Accordingly, down-regulating ATF6 level, thereby reducing ER stress signaling, by targeting its interactions with WFS1 and/or HRD1 is a potential new therapeutic method for treating ER stress disorders.

An inducible lentivirus expressing human WFS1 was generated. In brief, a human WFS1 was inserted into lentiviral expression vectors (pLenti CMV/TO; Invitrogen). Lentiviral particles were produced in 293T cells by transfection using Lipofectamine-2000. Lentiviral-containing supernatant was collected 48 hr after transfection and stored at −80° C. To establish a cell line that constitutively expressed the tetracycline repressor, INS-1 832/13 cells were infected with pLenti-TetR, followed by blasticidine selection (a kind gift from Dr. Eric Campeau). These cells were then infected overnight with the inducible lentiviruses (pLenti-CMV/TO-WFS1). After letting cells recover in fresh medium for 24 hr, puromycine was added (21 μg/mL) to select for transfected cells. To induce WFS1 in INS-1 832/13 cells, 2 μg/ml of doxycycline was added to the medium, which was then incubated for 48 hr.

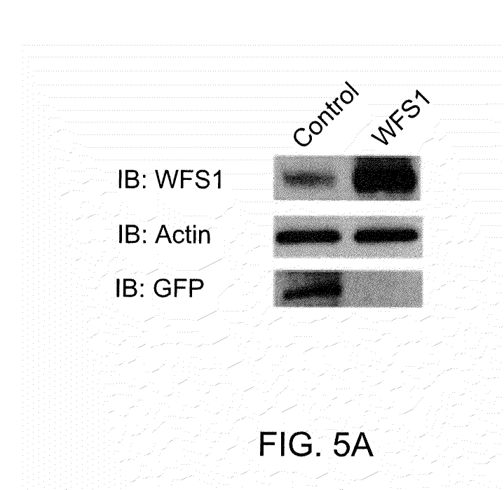

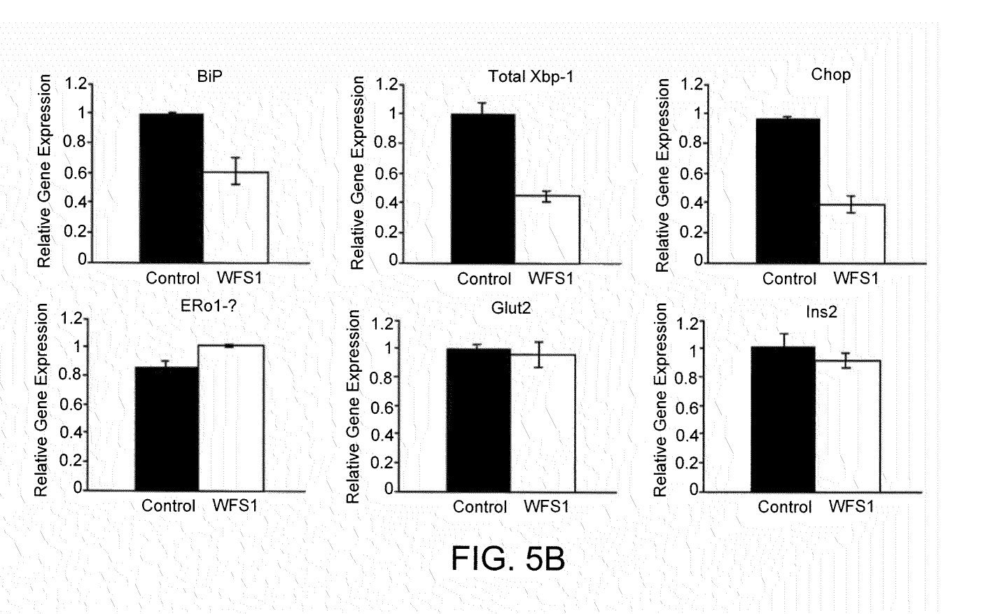

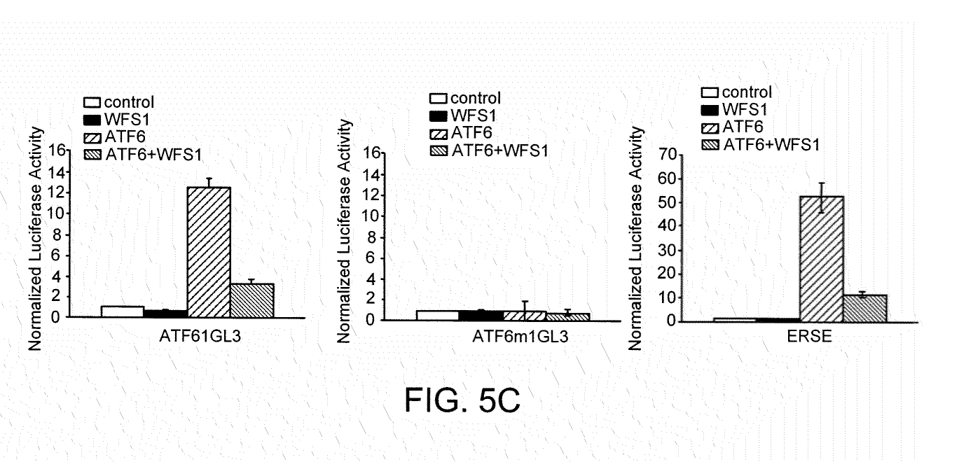

To examine whether WFS1 contributes to the regulation of ER stress signaling at the transcription level, total cell lysates were prepared from rat β-cell lines, INS-1 832/13, transduced with an inducible lentivirus expressing GFP (control) or human WFS1. The lysates were analyzed by immunoblot using an anti-WFS1 antibody, an anti-GFP antibody, and an antibody against actin as a loading control (FIG. 5A). Total mRNA was prepared from INS-1 832/13 cells over-expressing GFP (control) or WFS1, and expression levels of ER stress response genes, BiP, total Xbp-1, Chop, Ero-1α, Glut2, and Ins2, were measured by quantitative real-time PCR (n=3; values are mean±SD). It was found that expression levels of ER stress response genes, BiP, Chop, and Xbp-1, were decreased by 50% in cells over-expressing WFS1 as compared to control cells. However, gene expression levels of non-ER stress response genes, glucose transporter 2 (GLUT2), insulin 2 (INS2), and another ER stress response gene, endoplasmic reticulum oxidoreductin 1-alpha (Ero1-α) did not change by WFS1 expression (FIG. 5B).

ATF6 is a mediator of transcriptional induction of the ER stress response genes such as BiP and Chop (see K. Yamamoto et al., Dev Cell 13, 365 (2007); J. Wu et al., Dev Cell 13, 351 (2007)). To study if WFS1 directly regulates expression levels of ATF6 target genes by regulating ATF6 transcriptional activity, COS7 cells were transfected with ATF6 expression plasmid or ATF6 and WFS1 expression plasmids together with the ATF6 binding site reporter gene, ATF6GL3. This reporter was induced 12-fold by ATF6 and this induction was reduced to 3-fold by co-transfection of WFS1 (FIG. 5C, left panel). To confirm the specificity of activation of the ATF6 binding site, cells were transfected with ATF6 or ATF6 and WFS1 with the ATF6 mutant site reporter gene, ATF6m1GL3. This reporter was not induced by ATF6 or ATF6 and WFS1 (FIG. 5C, middle panel). It has been shown that ATF6 strongly activates the BiP/GRP78 promoter. Cells were also transfected with ATF6 or ATF6 and WFS1 with a rat GRP78 promoter reporter gene containing ER stress response element (ERSE). This reporter was induced more than 50-fold by ATF6 and this induction was reduced to 10-fold by co-transfection with WFS1 (FIG. 5C, right panel). Collectively, these results indicate that WFS1 suppresses the UPR at the transcription level.

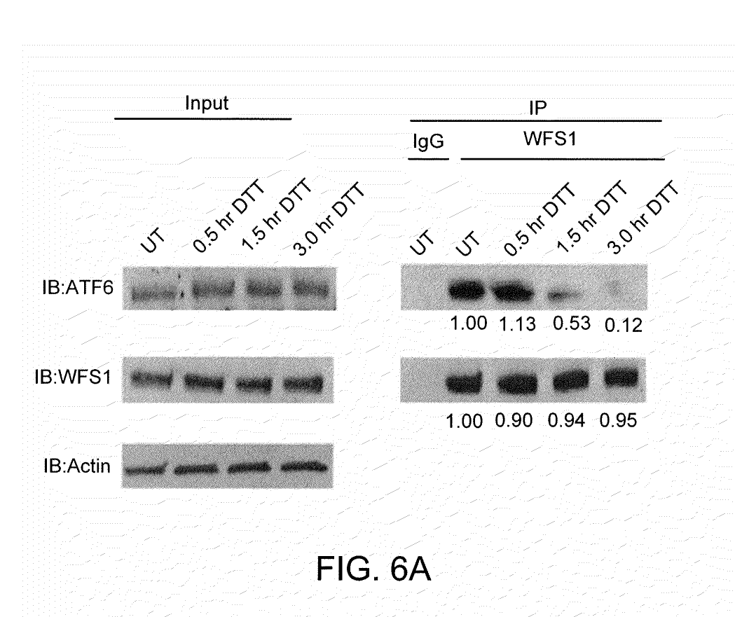

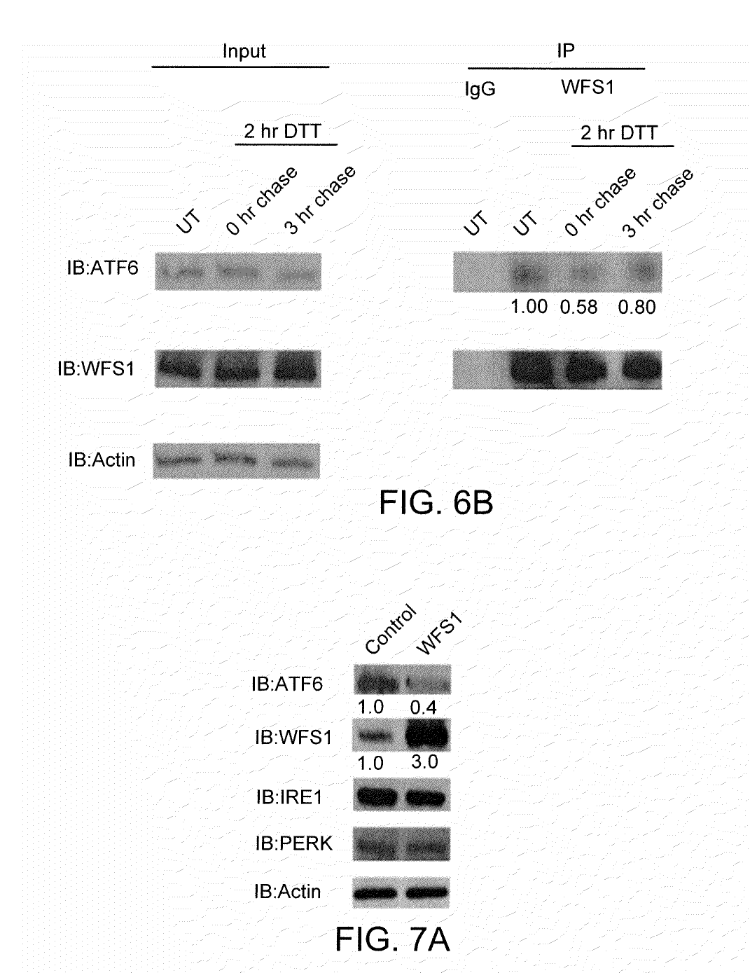

Both WFS1 and ATF6 are transmembrane proteins localized to the ER. The association of WFS1 with ATF6 in INS-1 832/13 cells was examined. An anti-WFS1 antibody was used to immunoprecipitate (IP) WFS1 from INS-1 832/13 cells untreated (UT) or treated with the ER stress inducer DTT (1 mM) for 0.5 hr, 1.5 hr, or 3 hr. Immunoprecipitates were then subject to immunoblot (IB) analysis using anti-ATF6, anti-WFS1, and anti-actin antibodies. FIG. 6A (left panel) shows that WFS1 associated with ATF6 under non-stress conditions. As shown in FIG. 6A (right panel), DTT treatment of INS-1 832/13 cells caused a dissociation of ATF6 from WFS1 in a time-dependent manner, with almost complete dissociation 3 hours post-treatment. To confirm that the interaction between WFS1 and ATF6 is recovered post-stress, an anti-WFS1 antibody was used to immunoprecipiate (IP) WFS1 from INS-1 832/13 cells untreated (UT) or treated with the ER stress inducer DTT (1 mM) for 2 hr. The cells were then chased in normal media for 0 hr and 3 hr. Immunoprecipitates were subject to immunoblot (IB) analysis using anti-ATF6, anti-WFS1, and anti-actin antibodies. The relative amount of ATF6 protein was quantified using ImageJ software. As shown in FIG. 6B, the interaction of ATF6 and WFS1 began to recover after a 3 hour chase. Together, these results show that WFS1 and ATF6 make a complex in an ER stress-dependent manner.

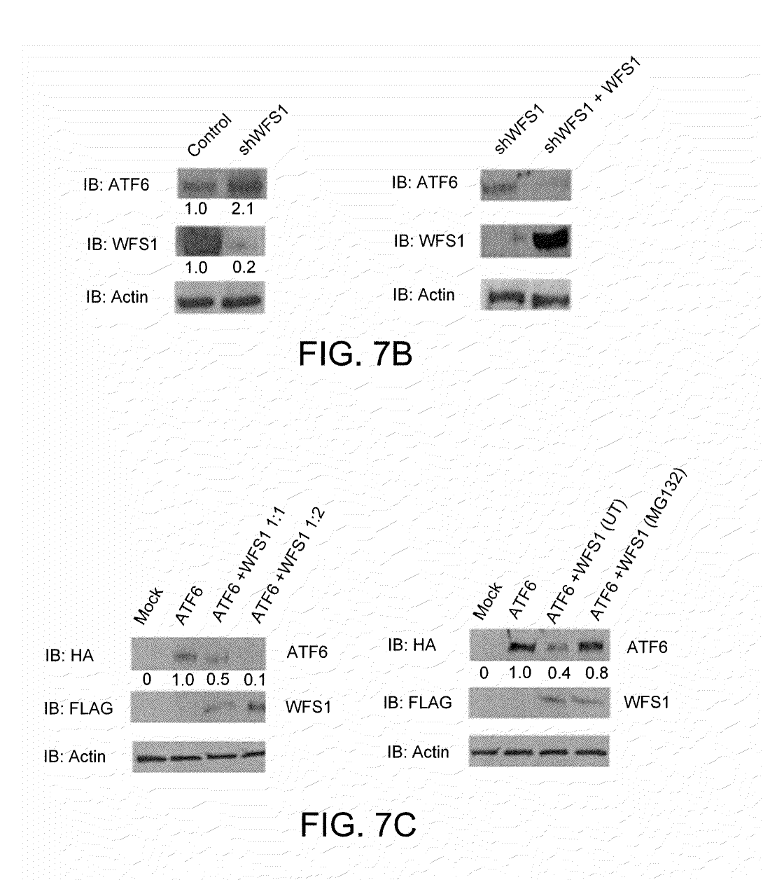

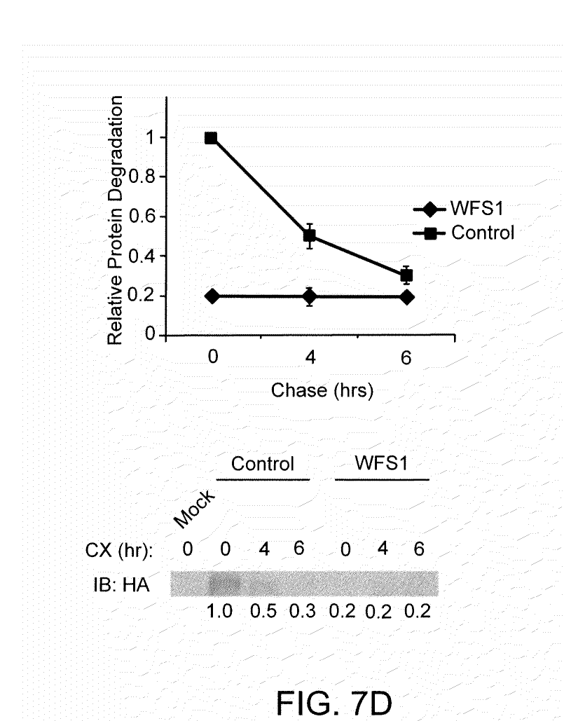

Whether WFS1 regulates steady-state expression levels of ATF6 protein and other UPR transducers was investigated. Whole cell lysates from INS-1 832/13 cells overexpressing GFP (control) or WFS1 were analyzed by immunoblot (IB) using anti-ATF6, anti-WFS1, anti-IRE 1, anti-PERK, and anti-actin antibodies. FIG. 7A shows that ATF6 protein level in the cells expressing WFS1 was reduced by more than 2-fold. As shown in FIG. 7A, the protein expression of the two other UPR transducers, IRE 1 and PERK, were not affected by WFS1 expression.

The relationship between WFS1 expression and ATF6 protein expression was further examined. Total cell lysates were prepared from mouse 1-cell lines, MIN6, transduced with a stable retrovirus expressing shRNA against GFP (control) or mouse WFS1, and analyzed by immunoblot using anti-WFS1 and anti-ATF6 antibodies and an antibody against actin as a loading control. MIN6 cells expressing shWFS1 or expressing shWFS1 and rescued with a WFS1 expression plasmid were immunoblotted with anti-WFS1 and anti-ATF6 antibodies, with anti-actin as a control. FIG. 7B (left panel) shows that ATF6 protein levels were increased approximately 2-fold compared to control MIN6 cells expressing shRNA directed against GFP. FIG. 7B (right panel) shows that ATF6 protein expression levels were again reduced when WFS1 was reintroduced.

Further, COS7 cells were transfected with ATF6-HA, or ATF6-HA and WFS1-FLAG at a 1:1 or 1:2 ratio of ATF6:WFS1. Whole cell extracts were then subject to immunoblot (IB) using anti-HA, anti-FLAG, and anti-actin antibodies. As shown in FIG. 7C (left panel), when WFS1 was expressed with ATF6 in a 1:1 ratio in COS-7 cells, the steady-state level of ATF6 protein was reduced by 2-fold, while a 1:2 ratio of ATF6 to WFS1 almost abolished ATF6 protein levels.

To investigate whether the decrease in ATF6 protein level is proteosome-dependent, COS7 cells expressing ATF6-HA or ATF6-HA and WFS1-FLAG were either untreated (UT) or treated with the proteosome inhibitor MG132 (15 μM) for 3 hr. Lysates were immunoblotted with anti-HA, anti-FLAG, and anti-actin antibodies. FIG. 7C (right panel) shows that treatment with MG132 led to an almost full recovery of ATF6 protein levels, suggesting that WFS1 enhances ATF6 degradation.

ATF6 stability was measured by determining its protein expression at various time points after treatment with the protein synthesis inhibitor cyclohexamide. COS7 cells transfected with ATF6-HA expression plasmid (control) or ATF6-HA together with WFS1-FLAG expression plasmids (WFS1) were treated with 40 μM cyclohexamide (CX) for 0 hr, 4 hr, and 6 hr. Whole cell lysates were subject to immunoblot (IB) with an anti-HA antibody. FIG. 7D shows that co-transfection of WFS1 with ATF6 decreased ATF6 protein expression levels as compared to control.

Whether WFS1 expression enhances ATF6 ubiquitination was examined. ATF6 was immunoprecipitated, using an anti-ATF6 antibody, from INS-1 832/13 cells overexpressing GFP (control) or WFS1 and treated with MG132 (0.1 μM) O/N. Immunoprecipitates were immunoblotted with anti-ubiquitin and anti-ATF6 antibodies. The relative amounts of ATF6 and WFS1 proteins were quantified using ImageJ software. As shown in FIG. 7E, when endogenous ATF6 was immunoprecipitated from INS-1 832/13 cells infected with lentivirus expressing human WFS1 or GFP and then treated with the protesome-inhibitor, MG132, ATF6 ubiquitination was enhanced in cells expressing WFS1. These results indicate that WFS1 has a function in the degradation of ATF6 through the ubiquitin-proteasome pathway.

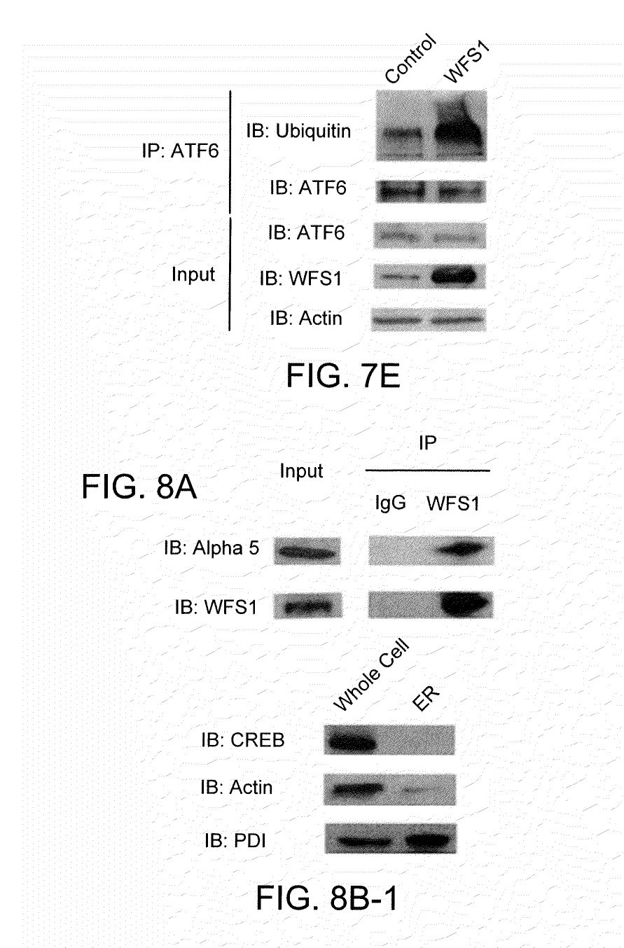

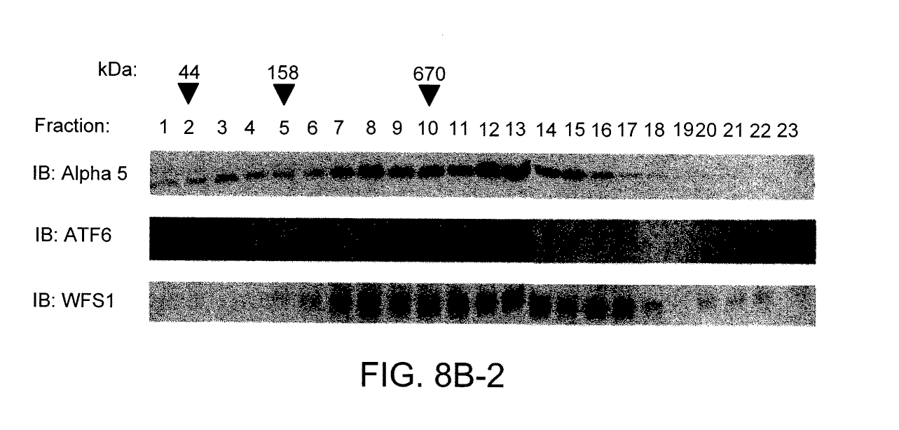

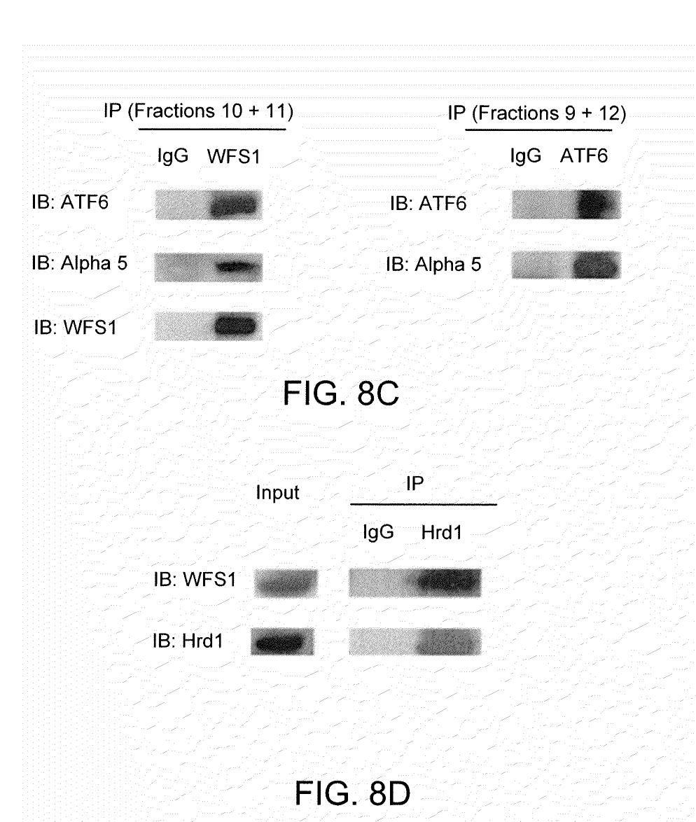

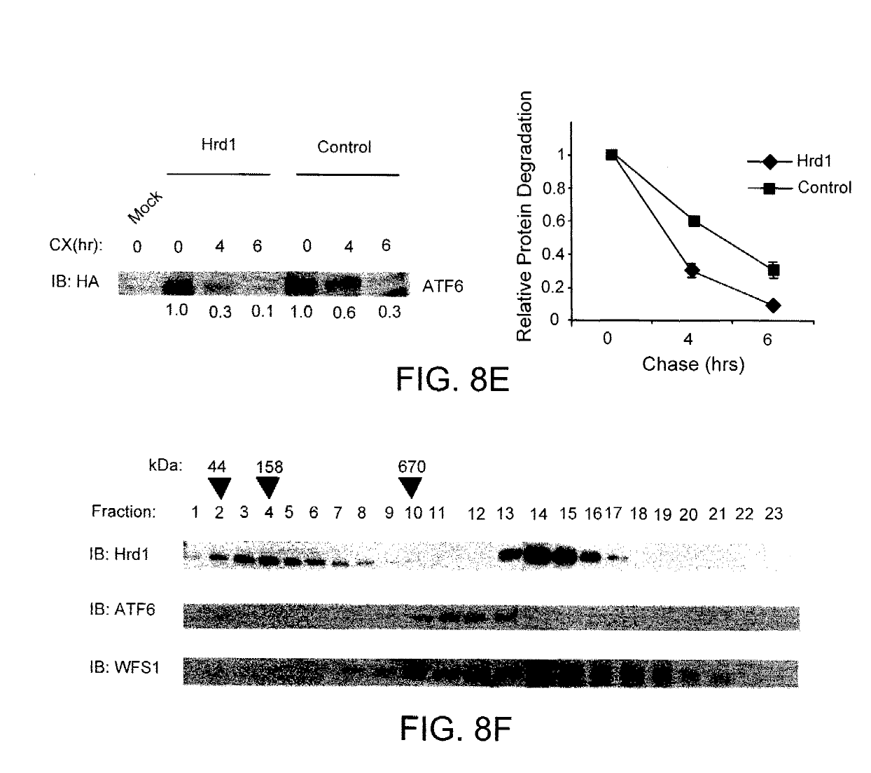

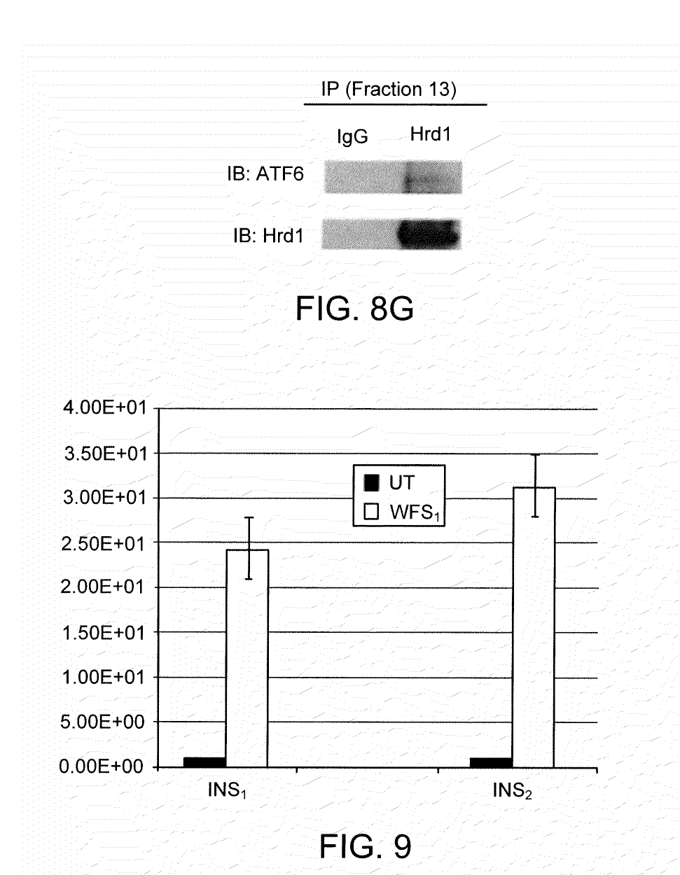

The ability of WFS1 to enhance the ubiquitination and degradation of ATF6 raised the possibility that WFS1 interacts with proteosome subunits and recruits the proteasome to ATF6 for degradation. To test this model, WFS1 was immunopreciptated from INS1 832/13 cells. The IP products were then immunoblotted with an α-5 proteasome subunit-specific antibody. FIG. 8A shows that WFS1 interacts with this proteasome subunit. To further study the formation of an ATF6-WFS1-proteasome complex, ER extracts were purified from INS-1 832/13 cells followed by fractionation using glycerol gradient sedimentation. Whole cell lysates or ER-isolated lysates of INS1 832/13 cells were subject to immunoblot (IB) using anti-CREB, anti-actin, and anti-PDI antibodies (FIG. 8B-1). ER-isolated lysates of INS1 832/13 cells were subject to fractionation using a 10-40% glycerol gradient. Fractions were analyzed by immunoblot using anti-alpha 5 20 s proteosome, anti-ATF6, and anti-WFS1 antibodies. The expression of the 26 S proteasome, ATF6, and WFS1 was found to overlap in fractions 8-13 (FIG. 8B-2). When WFS1 was immunoprecipitated from fractions 10-11, an interaction was found between WFS1 and ATF6, as well WFS1 and the proteasome (FIG. 8C, left panel). When ATF6 was immunoprecipitated from a mixture of factions 9 and 12, an ATF6-proteosome complex could be seen (FIG. 8C, right panel). These results indicate that WFS1, ATF6, and proteasome form a complex on the ER membrane.

Based on a homology search, WFS1 has a homology to an integral membrane protein of the ER, SEL1/HRD3, which has an important function in 3-hydroxy-3-methylglutaryl-CoA reductase (HMG-R) degradation (see R. Y. Hampton, R. G Gardner, J. Rine, Mol Biol Cell 7, 2029 (1996)). SEL1/HRD3 has been shown to interact with and stabilize the E3 ligase HRD1 (see, R. G Gardner et al., J Cell Biol 151, 69 (2000)). Whether WFS1 interacts with HRD1 was investigated. HRD1 was immunmoprecipated from INS1 832/13 lysates. The IP products were then immunoblotted with a WFS1-specific antibody. FIG. 8D shows that WFS1 and HRD1 form a complex.

Whether HRD1 would mark ATF6 for degradation by the proteasome was examined. 293T cells were transfected with an ATF6 expression plasmid or co-transfected with ATF6 and HRD1 expression plasmids, then ATF6 stability was measured by determining the expression levels of protein at various time points after treatment with the protein synthesis inhibitor cyclohexamide. The relative amount of ATF6 protein was quantified using ImageJ software. FIG. 8E shows that co-transfection of HRD1 with ATF6 enhanced ATF6 protein degradation as compared to control cells.

Whether WFS1, HRD1, and ATF6 form a complex on the ER membrane was determined. ER-isolated lysates of INS1 832/13 cells were subject to fractionation using a 10-40% glycerol gradient. Fractions were analyzed by immunoblot using anti-Hrd1, anti-ATF6, and anti-WFS1 antibodies. FIG. 8F shows that ATF6, HRD1, and WFS1 protein expression overlapped in fraction 13. When HRD1 was immunoprecipitated from this fraction, an interaction between ATF6 and HRD1 could be seen (FIG. 8G). Together, these data show that WFS1, ATF6 and HRD1 form a complex in an ER stress-dependent manner, and that HRD1 is an E3 ligase for ATF6.

### Example 3

### Insulin-Producing Exocrine Pancreatic Cells

In this example, evidence show that up-regulating the expression of WFS1 in exocrine pancreatic cells, e.g., acinar cells, which do not express WFS1 or produce insulin endogenously, can turn them into insulin producing cells.

Exocrine pancreatic cells, AR42J cells, were transfected with the inducible lentivirus expression vector that expressed human WFS1 described above. As shown in FIG. 9, production of insulin (INS1 and TNS2) was markedly increased in cells transfected with WFS1-expressing vector (WFS1) as compared to cells that were not transfected with the vector (UT). These results suggest that up-regulating WFS1 expression in non-insulin producing cells, e.g., exocrine pancreatic cells, can turn them into insulin-producing cells.

### Example 4

### Patients with WFS1 Mutations have a Higher Expression of Atf6

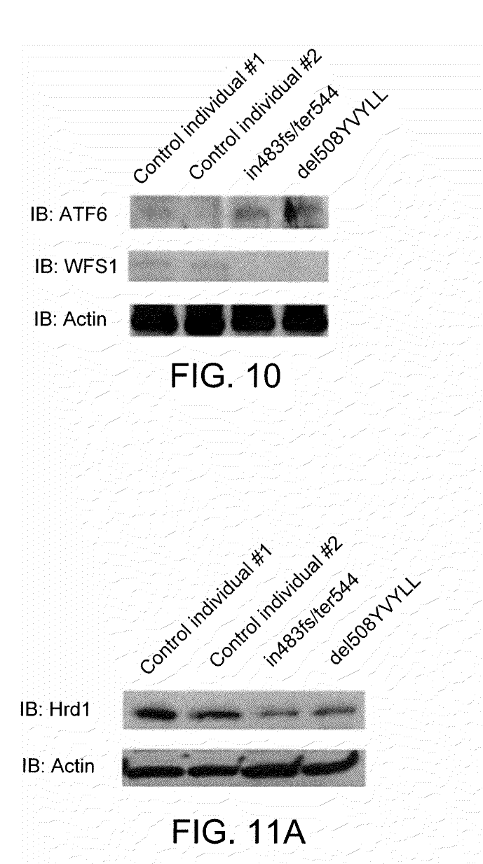

Lymphoblast lysates from Wolfram syndrome patients (ins483fs/ter544 and del508YVYLL) and control individuals were immunoblotted with anti-ATF6, anti-WFS1, and anti-actin antibodies. In samples from patients with WFS1 mutations, there was a higher expression of ATF6 protein, as compared with control samples (FIG. 10).

### Example 5

### Patients with WF51 Mutations have a Lower Expression of HRD1

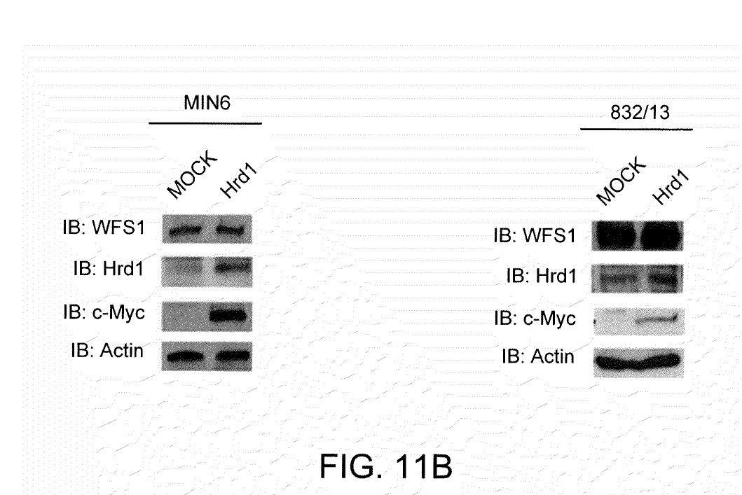

Lymphoblast lysates from Wolfram syndrome patients (ins483fs/ter544 and del508YVYLL) and control individuals were immunoblotted (IB) with anti-Hrd1 and anti-actin antibodies (n=3). In samples from patients with Wolfram syndrome, there was less HRD1 protein expression compared to control samples (FIG. 11A).

MIN6 cells were mock transfected or transfected with a Hrd1-Myc expression plasmid and lysates were subject to immunoblotting using anti-WFS1, anti-Hrd1, anti-c-Myc, and anti-actin antibodies (left panel). INS1 832/13 cells were mock transfected or transfected with a Hrd1-Myc expression plasmid and lysates were subject to IB using anti-WFS1, anti-Hrd1, anti-c-Myc, and anti-actin antibodies (right panel) (n=3). HRD1 expression did not affect WFS1 protein expression (FIG. 11B).

What is claimed is:

1. A method of determining a subject's risk of developing a condition associated with endoplasmic reticulum (ER) stress-related cell death, the method comprising:
providing a sample comprising a cell from the subject;
determining levels of one or both of HMG-CoA reductase degradation protein 1 (HRD1) and activating transcription factor 6 (ATF6) protein, or cellular localization of ATF6 protein in the sample; and
comparing the levels of one or both of HRD1 and ATF6 protein, or cellular localization of ATF6 protein in the sample with the corresponding levels of HRD1 and ATF6 protein, or cellular localization of ATF6 protein, in a control sample;
wherein a difference in the level of HRD1 or ATF6 protein, or cellular localization of ATF6, in the test sample as compared to the control sample indicates the subject's risk of developing a condition associated with ER stress-related cell death.

2. A method of treating a subject having a condition associated with endoplasmic reticulum stress-related cell death, the method comprising:
selecting a subject in need of such treatment; and
administering to the subject a therapeutically effective amount of one or more of:
an HRD1 protein, or a nucleic acid sequence encoding HRD1 protein; or
an ATF6-specific inhibitory nucleic acid or antagonist;
thereby treating the subject.

3. A method for identifying a candidate compound to treat a condition associated with endoplasmic reticulum (ER) stress-related cell death, the method comprising:
providing a cell expressing HRD1 and ATF6, wherein the cell expresses no or little Wolfram syndrome 1 homolog (WFS1) protein;
exposing the cell to a test compound; and
comparing protein levels of HRD1 and ATF6 in the cell in the presence of the test compound with levels of HRD1 and ATF6 in the absence of the test compound;
wherein a higher level of HRD1 or a lower level of ATF6 in the presence of the test compound than in its absence indicates that the test compound is a candidate compound for treating a disorder associated with ER stress-related cell death.

4. The method of claim 1, wherein the condition is diabetes mellitus, Parkinson's disease, optic atrophy, or amyotrophic lateral sclerosis.

5. The method of claim 2, wherein the condition is diabetes mellitus, Parkinson's disease, optic atrophy, or amyotrophic lateral sclerosis.

6. The method of claim 3, wherein the condition is diabetes mellitus, Parkinson's disease, optic atrophy, or amyotrophic lateral sclerosis.

7. The method of claim 1, wherein the cell is a lymphocyte, a pancreatic beta cell, or a neuron.

8. The method of claim 3, wherein the cell is a lymphocyte, a pancreatic beta cell, or a neuron.

9. The method of claim 3, wherein the cell is from a subject who has Wolfram Syndrome or diabetes mellitus.

10. The method of claim 1, wherein the control sample represents a level in a subject with a normal risk of developing a condition associated with ER stress-related cell death, and a decrease in HRD1 levels, an increase in ATF6 levels, or an increase in nuclear localization of ATF6, indicates that the subject has an increased risk of developing a condition associated with ER stress-related cell death.

11. A method for identifying a candidate compound for reducing endoplasmic reticulum (ER) stress-induced signaling, the method comprising:
providing a sample comprising HRD1 and ATF6 proteins;
contacting the sample with a test compound; and
comparing binding between HRD1 and ATF6 in the presence of the test compound with binding between HRD1 and ATF6 in the absence of the test compound;
wherein a higher level of binding in the presence of the test compound than in its absence indicates that the test compound is a candidate compound for reducing ER stress signaling.

12. The method of claim 11, wherein one or both of HRD1 and ATF6 are labeled.

13. The method of claim 11, wherein one or both of HRD1 and ATF6 are bound to a solid support.
